SEQUENCE LISTING (11 sequences)

SEQ ID NO: 1 - DNA, 10998 bp, Artificial Sequence
  Description of Artificial Sequence Synthetic nucleotide construct pONY8Z sequence
agatcttgaataataaaatgtgtgtttgtccgaaatacgcgttttgagatttctgtcgccgactaaattcatgtcgcgcgatagtggtgtttatcgccgatagagatggcgatattggaaaaattgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcgtaatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgggcactcagattctgcggtctgagtcccttctctgctgggctgaaaaggcctttgtaataaatataattctctactcagtccctgtctctagtttgtctgttcgagatcctacagttggcgcccgaacagggacctgagaggggcgcagaccctacctgttgaacctggctgatcgtaggatccccgggacagcagaggagaacttacagaagtcttctggaggtgttcctggccagaacacaggaggacaggtaagattgggagaccctttgacattggagcaaggcgctcaagaagttagagaaggtgacggtacaagggtctcagaaattaactactggtaactgtaattgggcgctaagtctagtagacttatttcatgataccaactttgtaaaagaaaaggactggcagctgagggatgtcattccattgctggaagatgtaactcagacgctgtcaggacaagaaagagaggcctttgaaagaacatggtgggcaatttctgctgtaaagatgggcctccagattaataatgtagtagatggaaaggcatcattccagctcctaagagcgaaatatgaaaagaagactgctaataaaaagcagtctgagccctctgaagaatatctctagaactagtggatcccccgggctgcaggagtggggaggcacgatggccgctttggtcgaggcggatccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcatgtacggtgggaggtctatataagcagagctcgtttagtgaaccgtcagatcgcctggagacgccatccacgctgttttgacctccatagaagacaccgggaccgatccagcctccgcggccccaagcttcagctgctcgaggatctgcggatccggggaattccccagtctcaggatccaccatgggggatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatccgcagatccggctgtggaatgtgtgtcagttagggtgtggaaagtccccaggctccccagcaggcagaagtatgcaaagcatgcctgcaggaattcgatatcaagcttatcgataccgtcgacctcgagggggggcccggtacccagcttttgttccctttagtgagggttaattgcgcgggaagtatttatcactaatcaagcacaagtaatacatgagaaacttttactacagcaagcacaatcctccaaaaaattttgtttttacaaaatccctggtgaacatgattggaagggacctactagggtgctgtggaagggtgatggtgcagtagtagttaatgatgaaggaaagggaataattgctgtaccattaaccaggactaagttactaataaaaccaaattgagtattgttgcaggaagcaagacccaactaccattgtcagctgtgtttcctgacctcaatatttgttataaggtttgatatgaatcccagggggaatctcaacccctattacccaacagtcagaaaaatctaagtgtgaggagaacacaatgtttcaaccttattgttataataatgacagtaagaacagcatggcagaatcgaaggaagcaagagaccaagaatgaacctgaaagaagaatctaaagaagaaaaaagaagaaatgactggtggaaaataggtatgtttctgttatgcttagcaggaactactggaggaatactttggtggtatgaaggactcccacagcaacattatatagggttggtggcgatagggggaagattaaacggatctggccaatcaaatgctatagaatgctggggttccttcccggggtgtagaccatttcaaaattacttcagttatgagaccaatagaagcatgcatatggataataatactgctacattattagaagctttaaccaatataactgctctataaataacaaaacagaattagaaacatggaagttagtaaagacttctggcataactcctttacctatttcttctgaagctaacactggactaattagacataagagagattttggtataagtgcaatagtggcagctattgtagccgctactgctattgctgctagcgctactatgtcttatgttgctctaactgaggttaacaaaataatggaagtacaaaatcatacttttgaggtagaaaatagtactctaaatggtatggatttaatagaacgacaaataaagatattatatgctatgattcttcaaacacatgcagatgttcaactgttaaaggaaagacaacaggtagaggagacatttaatttaattggatgtatagaaagaacacatgtattttgtcatactggtcatccctggaatatgtcatggggacatttaaatgagtcaacacaatgggatgactgggtaagcaaaatggaagatttaaatcaagagatactaactacacttcatggagccaggaacaatttggcacaatccatgataacattcaatacaccagatagtatagctcaatttggaaaagacctttggagtcatattggaaattggattcctggattgggagcttccattataaaatatatagtgatgtttttgcttatttatttgttactaacctcttcgcctaagatcctcagggccctctggaaggtgaccagtggtgcagggtcctccggcagtcgttacctgaagaaaaaattccatcacaaacatgcatcgcgagaagacacctgggaccaggcccaacacaacatacacctagcaggcgtgaccggtggatcaggggacaaatactacaagcagaagtactccaggaacgactggaatggagaatcagaggagtacaacaggcggccaaagagctgggtgaagtcaatcgaggcatttggagagagctatatttccgagaagaccaaaggggagatttctcagcctggggcggctatcaacgagcacaagaacggctctggggggaacaatcctcaccaagggtccttagacctggagattcgaagcgaaggaggaaacatttatgactgttgcattaaagcccaagaaggaactctcgctatcccttgctgtggatttcccttatggctattttggggactagtaattatagtaggacgcatagcaggctatggattacgtggactcgctgttataataaggatttgtattagaggcttaaatttgatatttgaaataatcagaaaaatgcttgattatattggaagagctttaaatcctggcacatctcatgtatcaatgcctcagtatgtttagaaaaacaaggggggaactgtggggtttttatgaggggttttataaatgattataagagtaaaaagaaagttgctgatgctctcataaccttgtataacccaaaggactagctcatgttgctaggcaactaaaccgcaataaccgcatttgtgacgcgagttccccattggtgacgcgttaacttcctgtttttacagtatataagtgcttgtattctgacaattgggcactcagattctgcggtctgagtcccttctctgctgggctgaaaaggcctttgtaataaatataattctctactcagtccctgtctctagtttgtctgttcgagatcctacagagctcatgccttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccaacctggcttatcgaaattaatacgactcactatagggagaccggc

SEQ ID NO: 2 - DNA, 8531 bp, Artificial Sequence
  Description of Artificial Sequence Synthetic nucleotide construct pONY8G sequence
agatcttgaataataaaatgtgtgtttgtccgaaatacgcgttttgagatttctgtcgccgactaaattcatgtcgcgcgatagtggtgtttatcgccgatagagatggcgatattggaaaaattgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcgtaatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgggcactcagattctgcggtctgagtcccttctctgctgggctgaaaaggcctttgtaataaatataattctctactcagtccctgtctctagtttgtctgttcgagatcctacagttggcgcccgaacagggacctgagaggggcgcagaccctacctgttgaacctggctgatcgtaggatccccgggacagcagaggagaacttacagaagtcttctggaggtgttcctggccagaacacaggaggacaggtaagattgggagaccctttgacattggagcaaggcgctcaagaagttagagaaggtgacggtacaagggtctcagaaattaactactggtaactgtaattgggcgctaagtctagtagacttatttcatgataccaactttgtaaaagaaaaggactggcagctgagggatgtcattccattgctggaagatgtaactcagacgctgtcaggacaagaaagagaggcctttgaaagaacatggtgggcaatttctgctgtaaagatgggcctccagattaataatgtagtagatggaaaggcatcattccagctcctaagagcgaaatatgaaaagaagactgctaataaaaagcagtctgagccctctgaagaatatctctagaactagtggatcccccgggctgcaggagtggggaggcacgatggccgctttggtcgaggcggatccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcatgtacggtgggaggtctatataagcagagctcgtttagtgaaccgtcagatcgcctggagacgccatccacgctgttttgacctccatagaagacaccgggaccgatccagcctccgcggccccaagcttgttgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagagtcgacctgcaggcatgcaagcttcagctgctcgagggggggcccggtacccagcttttgttccctttagtgagggttaattgcgcgggaagtatttatcactaatcaagcacaagtaatacatgagaaacttttactacagcaagcacaatcctccaaaaaattttgtttttacaaaatccctggtgaacatgattggaagggacctactagggtgctgtggaagggtgatggtgcagtagtagttaatgatgaaggaaagggaataattgctgtaccattaaccaggactaagttactaataaaaccaaattgagtattgttgcaggaagcaagacccaactaccattgtcagctgtgtttcctgacctcaatatttgttataaggtttgatatgaatcccagggggaatctcaacccctattacccaacagtcagaaaaatctaagtgtgaggagaacacaatgtttcaaccttattgttataataatgacagtaagaacagcatggcagaatcgaaggaagcaagagaccaagaatgaacctgaaagaagaatctaaagaagaaaaaagaagaaatgactggtggaaaataggtatgtttctgttatgcttagcaggaactactggaggaatactttggtggtatgaaggactcccacagcaacattatatagggttggtggcgatagggggaagattaaacggatctggccaatcaaatgctatagaatgctggggttccttcccggggtgtagaccatttcaaaattacttcagttatgagaccaatagaagcatgcatatggataataatactgctacattattagaagctttaaccaatataactgctctataaataacaaaacagaattagaaacatggaagttagtaaagacttctggcataactcctttacctatttcttctgaagctaacactggactaattagacataagagagattttggtataagtgcaatagtggcagctattgtagccgctactgctattgctgctagcgctactatgtcttatgttgctctaactgaggttaacaaaataatggaagtacaaaatcatacttttgaggtagaaaatagtactctaaatggtatggatttaatagaacgacaaataaagatattatatgctatgattcttcaaacacatgcagatgttcaactgttaaaggaaagacaacaggtagaggagacatttaatttaattggatgtatagaaagaacacatgtattttgtcatactggtcatccctggaatatgtcatggggacatttaaatgagtcaacacaatgggatgactgggtaagcaaaatggaagatttaaatcaagagatactaactacacttcatggagccaggaacaatttggcacaatccatgataacattcaatacaccagatagtatagctcaatttggaaaagacctttggagtcatattggaaattggattcctggattgggagcttccattataaaatatatagtgatgtttttgcttatttatttgttactaacctcttcgcctaagatcctcagggccctctggaaggtgaccagtggtgcagggtcctccggcagtcgttacctgaagaaaaaattccatcacaaacatgcatcgcgagaagacacctgggaccaggcccaacacaacatacacctagcaggcgtgaccggtggatcaggggacaaatactacaagcagaagtactccaggaacgactggaatggagaatcagaggagtacaacaggcggccaaagagctgggtgaagtcaatcgaggcatttggagagagctatatttccgagaagaccaaaggggagatttctcagcctggggcggctatcaacgagcacaagaacggctctggggggaacaatcctcaccaagggtccttagacctggagattcgaagcgaaggaggaaacatttatgactgttgcattaaagcccaagaaggaactctcgctatcccttgctgtggatttcccttatggctattttggggactagtaattatagtaggacgcatagcaggctatggattacgtggactcgctgttataataaggatttgtattagaggcttaaatttgatatttgaaataatcagaaaaatgcttgattatattggaagagctttaaatcctggcacatctcatgtatcaatgcctcagtatgtttagaaaaacaaggggggaactgtggggtttttatgaggggttttataaatgattataagagtaaaaagaaagttgctgatgctctcataaccttgtataacccaaaggactagctcatgttgctaggcaactaaaccgcaataaccgcatttgtgacgcgagttccccattggtgacgcgttaacttcctgtttttacagtatataagtgcttgtattctgacaattgggcactcagattctgcggtctgagtcccttctctgctgggctgaaaaggcctttgtaataaatataattctctactcagtccctgtctctagtttgtctgttcgagatcctacagagctcatgccttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccaacctggcttatcgaaattaatacgactcactatagggagaccggc

SEQ ID NO: 3 - protein, 524 aa, Rabies virus
MVPQALLFVPLLVFPLCFGKFPIYTIPDKLGPWSPIDIHHLSCPNNLVVEDEGCTNLSGFSYMELKVGYILAIKMNGFTCTGVVTEAETYTNFVGYVTTTFKRKHFRPTPDACRAAYNWKMAGDPRYEESLHNPYPDYRWLRTVKTTKESLVIISPSVADLDPYDRSLHSRVFPSGKCSGVAVSSTYCSTNHDYTIWMPENPRLGMSCDIFTNSRGKRASKGSETCGFVDERGLYKSLKGACKLKLCGVLGLRLMDGTWVAMQTSNETKWCPPDQLVNLHDFRSDEIEHLVVEELVRKREECLDALESIMTTKSVSFRRLSHLRKLVPGFGKAYTIFNKTLMEADAHYKSVRTWNEILPSKGCLRVGGRCHPHVNGVFFNGIILGPDGNVLIPEMQSSLLQQHMELLESSVIPLVHPLADPSTVFKDGDEAEDFVEVHLPDVHNQVSGVDLGLPNWGKYVLLSAGALTALMLIIFLMTCCRRVNRSEPTQHNLRGTGREVSVTPQSGKIISSWESHKSGGETRL

SEQ ID NO: 4 - DNA, 1650 bp, Rabies virus
aggaaagatggttcctcaggctctcctgtttgtaccccttctggtttttccattgtgttttgggaaattccctatttacacgatcccagacaagcttggtccctggagcccgattgacatacatcacctcagctgcccaaacaatttggtagtggaggacgaaggatgcaccaacctgtcagggttctcctacatggaacttaaagttggatacatcttagccataaaaatgaacgggttcacttgcacaggcgttgtgacggaggctgaaacctacactaacttcgttggttatgtcacaaccacgttcaaaagaaagcatttccgcccaacaccagatgcatgtagagccgcgtacaactggaagatggccggtgaccccagatatgaagagtctctacacaatccgtaccctgactaccgctggcttcgaactgtaaaaaccaccaaggagtctctcgttatcatatctccaagtgtagcagatttggacccatatgacagatcccttcactcgagggtcttccctagcgggaagtgctcaggagtagcggtgtcttctacctactgctccactaaccacgattacaccatttggatgcccgagaatccgagactagggatgtcttgtgacatttttaccaatagtagagggaagagagcatccaaagggagtgagacttgcggctttgtagatgaaagaggcctatataagtctttaaaaggagcatgcaaactcaagttatgtggagttctaggacttagacttatggatggaacatgggtcgcgatgcaaacatcaaatgaaaccaaatggtgccctcccgatcagttggtgaacctgcacgactttcgctcagacgaaattgagcaccttgttgtagaggagttggtcaggaagagagaggagtgtctggatgcactagagtccatcatgacaaccaagtcagtgagtttcagacgtctcagtcatttaagaaaacttgtccctgggtttggaaaagcatataccatattcaacaagaccttgatggaagccgatgctcactacaagtcagtcagaacttggaatgagatcctcccttcaaaagggtgtttaagagttggggggaggtgtcatcctcatgtgaacggggtgtttttcaatggtataatattaggacctgacggcaatgtcttaatcccagagatgcaatcatccctcctccagcaacatatggagttgttggaatcctcggttatcccccttgtgcaccccctggcagacccgtctaccgttttcaaggacggtgacgaggctgaggattttgttgaagttcaccttcccgatgtgcacaatcaggtctcaggagttgacttgggtctcccgaactgggggaagtatgtattactgagtgcaggggccctgactgccttgatgttgataattttcctgatgacatgttgtagaagagtcaatcgatcagaacctacgcaacacaatctcagagggacagggagggaggtgtcagtcactccccaaagcgggaagatcatatcttcatgggaatcacacaagagtgggggtgagaccagactgtgaggactggccgtcctttcaacgatccaagtcctgaagatcacctccccttggggggttctttttaaaaa

SEQ ID NO: 5 - DNA, 8870 bp, Artificial Sequence
  Description of Artificial Sequence Synthetic nucleotide construct pONY8.1Z sequence
agatcttgaataataaaatgtgtgtttgtccgaaatacgcgttttgagatttctgtcgccgactaaattcatgtcgcgcgatagtggtgtttatcgccgatagagatggcgatattggaaaaattgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcgtaatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgggcactcagattctgcggtctgagtcccttctctgctgggctgaaaaggcctttgtaataaatataattctctactcagtccctgtctctagtttgtctgttcgagatcctacagttggcgcccgaacagggacctgagaggggcgcagaccctacctgttgaacctggctgatcgtaggatccccgggacagcagaggagaacttacagaagtcttctggaggtgttcctggccagaacacaggaggacaggtaagattgggagaccctttgacattggagcaaggcgctcaagaagttagagaaggtgacggtacaagggtctcagaaattaactactggtaactgtaattgggcgctaagtctagtagacttatttcatgataccaactttgtaaaagaaaaggactggcagctgagggatgtcattccattgctggaagatgtaactcagacgctgtcaggacaagaaagagaggcctttgaaagaacatggtgggcaatttctgctgtaaagatgggcctccagattaataatgtagtagatggaaaggcatcattccagctcctaagagcgaaatatgaaaagaagactgctaataaaaagcagtctgagccctctgaagaatatctctagaactagtggatcccccgggctgcaggagtggggaggcacgatggccgctttggtcgaggcggatccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcatgtacggtgggaggtctatataagcagagctcgtttagtgaaccgtcagatcgcctggagacgccatccacgctgttttgacctccatagaagacaccgggaccgatccagcctccgcggccccaagcttcagctgctcgaggatctgcggatccggggaattccccagtctcaggatccaccatgggggatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatccgcagatccggctgtggaatgtgtgtcagttagggtgtggaaagtccccaggctccccagcaggcagaagtatgcaaagcatgcctgcaggaattcgatatcaagcttatcgataccgtcgaattggaagagctttaaatcctggcacatctcatgtatcaatgcctcagtatgtttagaaaaacaaggggggaactgtggggtttttatgaggggttttataaatgattataagagtaaaaagaaagttgctgatgctctcataaccttgtataacccaaaggactagctcatgttgctaggcaactaaaccgcaataaccgcatttgtgacgcgagttccccattggtgacgcgttaacttcctgtttttacagtatataagtgcttgtattctgacaattgggcactcagattctgcggtctgagtcccttctctgctgggctgaaaaggcctttgtaataaatataattctctactcagtccctgtctctagtttgtctgttcgagatcctacagagctcatgccttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccaacctggcttatcgaaattaatacgactcactatagggagaccggc

SEQ ID NO: 6 - DNA, 24 bp, Unknown Organism
  Description of Unknown Organism Illustrative DNA sequence
atttacacgatactagacaagctt

SEQ ID NO: 7 - protein, 8 aa, Unknown Organism
  Description of Unknown Organism Illustrative amino acid sequence
IYTILDKL

SEQ ID NO: 8 - DNA, 24 bp, Artificial Sequence
  Description of Artificial Sequence Synthetic DNA sequence of the present invention
atttacacgatcccagacaagctt

SEQ ID NO: 9 - protein, 8 aa, Artificial Sequence
  Description of Artificial Sequence Synthetic amino acid sequence of the present invention
IYTIPDKL

SEQ ID NO: 10 - DNA, 24 bp, Artificial Sequence
  Description of Artificial Sequence Primer
cgttgctgcataaaccgactacac

SEQ ID NO: 11 - DNA, 22 bp, Artificial Sequence
  Description of Artificial Sequence Primer
tgcagaggatgatgctcgtgac

(19) United States
(12) Patent Application Publication
(10) Pub. No.: US 2004/0071675 A1
(43) Pub. Date: Apr. 15, 2004

(54) Vector system

(76) Inventors: Nicholas Mazarakis, Oxford (GB); Mimoun Azzouz, Oxford (GB)

Correspondence Address: FROMMER LAWRENCE & HAUG, 745 FIFTH AVENUE- 10TH FL., NEW YORK, NY, 10151, US

(21) Appl. No.: 10/429,608

(22) Filed: May 5, 2003

Related U.S. Application Data
(63) Continuation-in-part of application No. PCT/GB01/04866, filed on Nov. 2, 2001.

(30) Foreign Application Priority Data
Nov. 3, 2000 (GB) 0026943.1
Jan. 30, 2001 (GB) 0102339.9
Sep. 14, 2001 (GB) 0122238.9

(51) Int. Cl. A61K 48/00; C12N 5/08; C12N 15/85

(52) U.S. Cl. 424/93.21; 435/455; 435/368

(57) ABSTRACT

There is provided the use of a vector system comprising at least part of a rabies g protein, to transduce a TH positive neuron. There is also provided the use of a rabies G vector system to transduce a target site, in which the vector system travels to the target site by retrograde transport, which may comprise the step of administration of the vector system to an administration site which is distant from the target site.

### FIELD OF THE INVENTION

The present invention relates to-a vector system. In particular, the present invention relates to a vector system capable of delivering an entity of interest (EOI)—such as a nucleotide sequence of interest (“NOI”)—to a neuron.

In one preferred aspect, the present invention relates to a viral vector system capable of delivering an EOI to a TH positive neuron, such as for the treatment of Parkinson's disease.

In another preferred aspect, the present invention relates to a vector system capable of travelling to a target site by retrograde transport. In particular, the present invention relates to the use of such a vector system to transduce distal connected sites within the nervous system. The vector system may be administered peripherally, for example by peripheral intramuscular delivery.

### BACKGROUND TO THE INVENTION

Parkinson's Disease

Although the cause of Parkinson's disease is not known, it is associated with the progressive death of dopaminergic (tyrosine hydroxylase (TH) positive) mesencephalic neurons, inducing motor impairment. The characteristic symptoms of Parkinson's disease appear when up to 70% of TH-positive nigrostriatal neurons have degenerated.

There is currently no satisfactory cure for Parkinson's disease. Symptomatic treatment of the disease-associated motor impairments involves oral administration of L-DOPA. L-DOPA is transported across the blood-brain barrier and converted to dopamine, partly by residual dopaminergic neurons, leading to a substantial improvement of motor function. However, after a few years, the degeneration of dopaminergic neurons progresses, the effects of L-DOPA are reduced and side-effects reappear. Better therapy for Parkinson's disease is therefore necessary.

An alternative strategy for therapy is neural grafting, which is based on the idea that dopamine supplied from cells implanted into the striatum can substitute for lost nigrostriatal cells. Clinical trials have shown that mesencephalic TH positive neurons obtained from human embryo cadavers (aborted foetuses) can survive and function in the brains of patients with Parkinson's disease. However, functional recovery has only been partial, and the efficacy and reproducibility of the procedure is limited. Also, there are ethical, practical and safety issues associated with using tissue derived from aborted human foetuses. Moreover, the large amounts of tissue required to produce a therapeutic effect is likely to prove to be prohibitive. Some attempts have been made to use TH positive neurons from other species (in order to circumvent some of the ethical and practical problems). However, xenotransplantation requires immunosuppressive treatment and is also controversial due to, for example, the possible risk of cross-species transfer of infectious agents. Another disadvantage is that, in current grafting protocols, no more than 5-20% of the expected numbers of grafted TH positive neurons survive. In order to develop a practicable and effective transplantation protocol, an alternative source of TH positive neurons is required.

A further alternative strategy for therapy is gene therapy. It has been suggested that gene therapy could be used in Parkinson's disease in two ways: to replace dopamine in the affected striatum by introducing the enzymes responsible for L-DOPA or dopamine synthesis (for example, tyrosine hydroxylase); and to introduce potential neuroprotective molecules that may either prevent the TH-positive neurons from dying or stimulate regeneration and functional recovery in the damaged nigrostriatal system (Dunnet S. B. and Bjorklund A. (1999) Nature 399 A32-A39).

However, although primary neuronal cultures from rat fetal ventral mesencephalon (which typically contain 4% TH positive neurons) have been transduced with hybrid adeno-associated virus (AAV)/herpes simplex virus (HSV) vectors (Constantini L. C. et al (1999) Human Gene Therapy 10:2481-2494), this was not demonstrated to reflect significant transduction of the TH positive sub-population. TH positive neurons have proved to be very refractory to transduction with MV vectors, HSV vectors, and hybrid HSV/AAV vectors. Adenovirus vectors have limited success only at very high mois (at a moi of 400, 40% transduction efficiency has been achieved) (Karen O'Malley personal communication). The in vivo transduction capabilities of these vectors for nigral dopaminergic neurons is also poor or not well characterised.

Another problem with gene therapy approaches in the treatment of Parkinson's disease, is that brain is a difficult and complex organ to target (Raymon H. K. et al (1997) Exp. Neur. 144: 82-91). The usual route is by injection of vectors to the striatum (Bilang-Bleuel et al (1997)-Proc. Acad. Natl. Sci. USA 94:8818-8823; Choi-Lundberg et al (1998) Exp. Neurol. 154:261-275) or to near the substantia nigra (Choi-Lundberg et al (1997) Science 275:838-841; Mandel et al (1997)) Proc. Acad. Natl. Sci. USA 94:14083-14088). It is technically difficult to inject directly into the some parts of the brain, for example because of their location and/or size. The substantia nigra lies deep in the brain and direct injection to this area can cause lesion of axons, resulting in damage. The striatum, (in particular the caudate putamen) is a relatively easy target because it is larger and more dorsal than the substantia nigra. It has been used extensively for transplantation in Parkinson's disease, and there is currently thought to be less than 1% risk involved in the operation.

Hence, it is desirable to find a mechanism for transducing parts of the brain which are difficult to reach by direct injection. It is also desirable to find an administration strategy for cranial gene therapy which minimises the number and complexity of brain injections.

It is also desirable to find a mechanism for transducing TH positive neurons.

It is also desirable to provide an alternative and improved source of TH positive neurons for transplantation.

Finally, it is desirable to provide a better therapeutic approach for the treatment and/or prevention of Parkinson's disease.

CNS Gene Therapy

In addition to Parkinson's disease, somatic cell gene transfer is a strategy which offers promise for the investigation and therapy of many genetic and degenerative disorders of the central nervous system (CNS). CNS gene therapy has been limited, however, by difficulties relating to gene delivery. These difficulties result from (a) the blood-brain barrier, a capillary barrier which allows relatively little transport of blood-borne molecules; (b) complex compartmentalisation of the CNS into distinct functional cellular groups and tracts; (c) vulnerability of critical CNS tissue to-direct injection with viruses and nucleic acids.

It is therefore desirable to provide a method for transducing cells within the CNS which (a) obviates the need to cross the blood-brain barrier, (b) is able to target the required group of cells, and (c) avoids damaging CNS tissue during the administration step.

In addition to CNS tissue being vulnerable to direct injection, it is awkward to access. In some cases direct access is not possible, for example in cases of suspected paraplegia, there is a three-week period following injury in which direct access to the spinal cord is impossible. During this period it is not possible to perform an epidural and so it is necessary to administer any drugs (for example pain-killers and anti-inflammatories) via the oral route.

It is therefore desirable to provide an alternative to direct injection for transducing cells in the CNS.

### SUMMARY OF ASPECTS OF THE PRESENT INVENTION

In a broad aspect, the present invention relates to a vector system that is capable of transporting an entity of interest (“EOI”).

As used herein the term “vector system” includes any vector that is capable of infecting or transducing or transforming or modifying a recipient cell with an EOI.

The vector system is or comprises at least a part of a rabies G protein or a mutant, variant, homologue or fragment thereof. Typically the vector system will also comprise an EOI.

The vector system can be a non-viral system or a viral system, or combinations thereof. In addition, the vector system itself can be delivered by viral or non-viral techniques.

In non-viral vector systems of the present invention, the at least part of the rabies G protein (or a mutant, variant, homologue or fragment thereof may be used to encapulate or enshroud an EOI. Thus, for some embodiments, the at least part of the rabies G protein (or a mutant, variant, homologue or fragment thereof) may form a matrix around the EOI. Here, the matrix may contain other components—such as a liposome type entity.

In some preferred aspects, the vector system is a viral vector system.

In some further preferred aspects, the vector system is a retroviral vector system.

If the vector system is a viral vector system, in particular a retroviral vector system, then typically the vector system is pseudotyped with at least a part of a rabies G protein or a mutant, variant, homologue or fragment thereof.

In a preferred aspect, it has been found that a particular type of vector system especially a viral vector system (e.g. a retroviral vector system)—is capable of transducing TH positive neurons, a subset of neurons which are notoriously refractory to transduction.

In one embodiment, the invention provides the use of a vector system—such as viral vector system, preferably a retroviral vector system—to transduce a TH positive neuron, in which the viral vector system is or comprises (such as is pseudotyped with) at least a part of a rabies G protein or a mutant, variant, homologue or fragment thereof.

It has also been found that a particular type of vector system—such as viral vector system, preferably a retroviral vector system—according to the present invention is capable of transducing one or more sites which are distant from the site of administration due to retrograde transport of the vector system.

In another embodiment, the present invention provides the use of a vector system, preferably a viral vector delivery system, more preferably a retroviral vector system, to transduce a target site, in which the vector system travels to the target site by retrograde transport, and in which the vector system is or comprises (such as is pseudotyped with) at least a part of a rabies G protein or a mutant, variant, homologue or fragment thereof.

There is also provided the use of a vector system—such as viral vector system, preferably a retroviral vector system—to transduce a target site, which comprises the step of administration of the retroviral vector system to an administration site which is distant from the target site, in which the retroviral vector system is or comprises (such as is pseudotyped with) at least a part of a rabies G protein or a mutant, variant, homologue or fragment thereof.

Administration to a single target site may cause transduction of a plurality of target sites. The vector system may travel to the or each target by retrograde transport, optionally in combination with anterograde transport.

In further broad aspects, the present invention relates to:

(i) a method of treating and/or preventing a diseases using such a vector system;

(ii) the use of such a vector system in the manufacture of a pharmaceutical composition to treat and/or prevent a disease;

(iii) a method for analysing the effect of a protein of interest in a cell using such a vector system;

(iv) a method for analysing the function of a gene or protein using such a vector system;

(v) a cell tranduced with such a vector system;

(vi) a genetically modified (for example immortalised) cell made by transduction with such a vector system;

(vii) the use of such a genetically modified (for example immortalised) cell in the manufacture of a medicament; and

(viii) a transplantation method using such a genetically modified (for example immortalised) cell.

Administration of the vector system to a site which is distant from the target site opens up the possibility of accessing target sites for which direct administration is problematic. There are a number of problems associated with accessing sites within the CNS by direct injection as alluded to above, which may be obviated using retrograde transport of the vector system.

In another embodiment, the invention provides a method for transducing a neuron in the CNS which comprises the following steps:

(i) administration of a vector system (such as viral vector system, preferably a retroviral vector system) to a peripheral site

(ii) retrograde transport of the vector system or part thereof to the neuron wherein the vector system is or comprises (such as is pseudotyped with) at least a part of a rabies G protein or a mutant, variant, homologue or fragment thereof.

### DETAILED DESCRIPTION OF THE INVENTION

The present invention relates to a new use of a vector system.

The vector system can be a non-viral system or a viral system.

Viral vector or viral delivery systems include but are not limited to adenoviral vectors, adeno-associated viral (AAV) vectors, herpes viral vectors, retroviral vectors, lentiviral vectors, and baculoviral vectors. Non-viral delivery or non-viral vector systems include lipid mediated transfection, liposomes, immunoliposomes, lipofectin, cationic facial amphiphiles (CFAs) and combinations thereof.

In some preferred aspects, the vector system is a viral vector system.

In some further preferred aspects, the vector system is a retroviral vector system.

Retroviruses

The concept of using viral vectors for gene therapy is well known (Verma and Somia (1997) Nature 389:239242).

There are many retroviruses. For the present application, the term “retrovirus” includes: murine leukemia virus (MLV), human immunodeficiency virus (HIV), equine infectious anaemia virus (EIAV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus 29 (MC29), and Avian erythroblastosis virus (AEV) and all other retroviridiae including lentiviruses.

A detailed list of retroviruses may be found in Coffin et al (“Retroviruses” 1997 Cold Spring-Harbour Laboratory Press Eds: J M Coffin, S M Hughes, H E Varmus-pp 758-763).

In a preferred embodiment, the retroviral vector system is derivable from a lentivirus. Lentiviruses also belong to the retrovirus family, but they can infect both dividing and non-dividing cells (Lewis et al (1992) EMBO J. 3053-3058).

The lentivirus group can be split into “primate” and “non-primate”. Examples of primate lentiviruses include the human immunodeficiency virus (HIV), the causative agent of human acquired immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV) and bovine immunodeficiency virus (BIV). In a preferred embodiment, the retroviral vector system is derivable from EIAV.

Details on the genomic structure of some lentiviruses may be found in the art. By way of example, details on HIV and EIAV may be found from the NCBI Genbank database (i.e. Genome Accession Nos. AF033819 and AF033820 respectively). Details of HIV variants may also be found at http://hiv-web.lanl.gov. Details of EIAV variants may be found through http://www.ncbi.nim.nih.gov.

During the process of infection, a retrovirus initially attaches to a specific cell surface receptor. On entry into the susceptible host cell, the retroviral RNA genome is then copied to DNA by the virally encoded reverse transcriptase which is carried inside the parent virus. This DNA is transported to the host cell nucleus where it subsequently integrates into the host genome. At this stage, it is typically referred to as the provirus. The provirus is stable in the host chromosome during cell division and is transcribed like other cellular genes. The provirus encodes the proteins and other factors required to make more virus, which can leave the cell by a process sometimes called “budding”.

Each retroviral genome comprises genes called gag, pol and env which code for virion proteins and enzymes. These genes are flanked at both ends by regions called long terminal repeats (LTRs). The LTRs are responsible for proviral integration, and transcription: They also serve as enhancer promoter sequences. In other words, the LTRs can control the expression of the viral genes. Encapsidation of the retroviral RNAs occurs by virtue of a psi sequence located at the 5′ end of the viral genome.

The LTRs themselves are identical sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

For the viral genome, the site of transcription initiation is at the boundary between U3 and R in one LTR and the site of poly (A) addition (termination) is at the boundary between R and U5 in the other LTR. U3 contains most of the transcriptional control elements of the provirus, which include the promoter and multiple enhancer sequences responsive to cellular and in some cases, viral transcriptional activator proteins. Some retroviruses have any one or more of the following genes that code for proteins that are involved in the regulation of gene expression: tat, rev, tax and rex.

With regard to the structural genes gag, pol and env themselves, gag encodes the internal structural protein of-the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome. The env gene encodes the surface (SU) glycoprotein and the transmembrane (TM) protein of the virion, which form a complex that interacts specifically with cellular receptor proteins. This interaction leads ultimately to infection by fusion of the viral membrane with the cell membrane.

Retroviruses may also contain additional genes which code for proteins other than gag, pol and env. Examples of additional genes include in HIV, one or more of vif, vpr, vpx, vpu, tat, rev and nef EIAV has (amongst others) the additional gene S2.

Proteins encoded by additional genes serve various functions, some of which may be duplicative of a function provided by a cellular protein. In EIAV, for example, tat acts as a transcriptional activator of the viral LTR. It binds to a stable, stem-loop RNA secondary structure referred to as TAR. Rev regulates and co-ordinates the expression of viral genes through rev-response elements (RRE). The mechanisms of action of these two proteins are thought to be broadly similar to the analogous mechanisms in the primate viruses. The function of S2 is unknown. In addition, an EIAV protein, Ttm, has been identified that is encoded by the first exon of tat spliced to the env coding sequence at the start of the transmembrane protein.

VECTOR SYSTEMS

The vector system can be a non-viral system or a viral system.

In some preferred aspects, the vector system is a viral vector system.

In some further preferred aspects, the vector system is a retroviral vector system.

The vector system can be used to transfer an EOI to one or more sites of interest. The transfer can occur in vitro, ex vivo, in vivo, or combinations thereof. In a highly preferred aspect, the delivery system is a retroviral delivery system. Retroviral vector systems have been proposed as a delivery system for inter alia the transfer of an EOI to one or more sites of interest. The transfer can occur in vitro ex vivo, in vivo, or combinations thereof. Retroviral vector systems have even been exploited to study various aspects of the retrovirus life cycle, including receptor usage, reverse transcription and RNA packaging (reviewed by Miller, 1992 Curr Top Microbiol Immunol 158:1-24).

As used herein the term “Vector system” also includes a vector particle capable of transducing a recipient cell with an NOI.

A vector particle includes the following components: a vector genome, which may contain one or more NOIs, a nucleocapsid encapsidating the nucleic acid, and a membrane surrounding the nucleocapsid.

The term “nucleocapsid” refers to at least the group specific viral core proteins (gag) and the viral polymerase (pol) of a retrovirus genome. These proteins encapsidate the packagable sequences and are themselves further surrounded by a membrane containing an envelope glycoprotein.

Once within the cell, the RNA genome from a retroviral vector particle is reverse transcribed into DNA and integrated into the DNA of the recipient cell.

The term “vector genome” refers to both to the RNA construct present in the retroviral vector particle and the integrated DNA construct. The term also embraces a separate or isolated DNA construct capable of encoding such an RNA genome. A retroviral or lentiviral genome should comprise at least one component part derivable from a retrovirus or a lentivirus. The term “derivable” is used in its normal sense as meaning a nucleotide sequence or a part thereof which need not necessarily be obtained from a virus such as a lentivirus but instead could be derived therefrom. By way of example, the sequence may be prepared synthetically or by use of recombinant DNA techniques. Preferably the genome comprises a psi region (or an analogous component which is capable of causing encapsidation).

The viral vector genome is preferably “replication defective” by which we mean that the genome does not comprise sufficient genetic information alone to enable independent replication to produce infectious viral particles within the recipient cell. In a preferred embodiment, the genome lacks a functional env, gag or pol gene. If a highly preferred embodiment the genome lacks env, gag and pol genes.

The viral vector genome may comprise some or all of the long terminal repeats (LTRs). Preferably the genome comprises at least part of the LTRs or an analogous sequence which is capable of mediating proviral integration, and transcription. The sequence may also comprise or act as an enhancer-promoter sequence.

It is known that the separate expression of the components required to produce a retroviral vector particle on separate DNA sequences cointroduced into the same cell will yield retroviral particles carrying defective retroviral genomes that carry therapeutic genes (e.g. Reviewed by Miller 1992). This cell is referred to as the producer cell (see below).

There are two common procedures for generating producer cells. In one, the sequences encoding retroviral Gag, Pol and Env proteins are introduced into the cell and stably integrated into the cell genome; a stable cell line is produced which is referred to as the packaging cell line. The packaging cell line produces the proteins required for packaging retroviral RNA but it cannot bring about encapsidation due to the lack of a psi region. However, when a vector genome (having a psi region) is introduced into the packaging cell line, the helper proteins can package the psi-positive recombinant vector RNA to produce the recombinant virus stock. This can be used to transduce the NOI into recipient cells. The recombinant virus whose genome lacks all genes required to make viral proteins can infect only once and cannot propagate. Hence, the NOI is introduced into the host cell genome without the generation of potentially harmful retrovirus. A summary of the available packaging lines is presented in “Retroviruses” (1997 Cold Spring Harbour Laboratory Press Eds: JM Coffin, SM Hughes, HE Varmus pp 449). The present invention also provides a packaging cell line comprising a viral vector genome which is capable of producing a vector system useful in the first aspect of the invention. For example, the packaging cell line may be transduced with a viral vector system comprising the genome or transfected with a plasmid carrying a DNA construct capable of encoding the RNA genome. The present invention also provides a kit for producing a retroviral vector system useful in the first aspect of the invention which comprises a packaging cell and a retroviral vector genome.

The second approach is to introduce the three different DNA sequences that are required to produce a retroviral vector particle i.e. the env coding sequences, the gag-pol coding sequence and the defective retroviral genome containing one or more NOIs into the cell at the same time by transient transfection and the procedure is referred to as transient triple transfection (Landau & Littman 1992; Pear et al 1993). The triple transfection procedure has been optimised (Soneoka et al 1995; Finer et al 1994). WO 9412-9438 describes the production of producer-cells in vitro using this multiple DNA transient transfection method. WO 97/27310 describes a set of DNA sequences for creating retroviral producer cells either in vivo or in vitro for re-implantation.

The components of the viral system which are required to complement the vector genome may be present on one or more “producer plasmids” for transfecting into cells.

The present invention also provides a kit for producing a retroviral vector system useful in the first aspect of the invention, comprising

(i) a viral vector genome which is incapable of encoding one or more proteins which are required to produce a vector particle;

(ii) one or more producer plasmid(s) capable of encoding the protein which is not encoded by (i); and optionally

(iii) a cell suitable for conversion into a producer cell.

In a preferred embodiment, the viral vector genome is incapable of encoding the proteins gag, pol and env. Preferably the kit comprises one or more producer plasmids encoding env, gag and pol, for example, one producer plasmid encoding env and one encoding gag-pol. Preferably the gag-pol sequence is codon optimised for use in the particular producer cell (see below).

The present invention also provides a producer cell expressing the vector genome and the producer plasmid(s) capable of producing a retroviral vector system useful in the present invention.

Preferably the retroviral vector system used in the first aspect of the present invention is a self-inactivating (SIN) vector system.

By way of example, self-inactivating retroviral vector systems have been constructed by deleting the transcriptional enhancers or the enhancers and promoter in the U3 region of the 3′ LTR. After a round of vector reverse transcription and integration, these changes are copied into both the 5′ and the 3′ LTRs producing a transcriptionally inactive provirus. However, any promoter(s) internal to the LTRs in such vectors will still be transcriptionally active. This strategy has been employed to eliminate effects of the enhancers and promoters in the viral LTRs on transcription from internally placed genes. Such effects include increased transcription or suppression of transcription. This strategy can also be used to eliminate downstream transcription from the 3′ LTR into genomic DNA. This is of particular concern in human gene therapy where it may be important to prevent the adventitious activation of an endogenous oncogene.

Preferably a recombinase assisted mechanism is used which facilitates the production of high titre regulated lentiviral vectors from the producer cells of the present invention.

As used herein, the term “recombinase assisted system” includes but is not limited to a system using the Cre recombinase/IoxP recognition sites of bacteriophage P1 or the site-specific FLP recombinase of S. cerevisiae which catalyses recombination events between 34 bp FLP recognition targets (FRTs). The site-specific FLP recombinase of S. cerevisiae which catalyses recombination events between 34 bp FLP recognition targets (FRTs) has been configured into DNA constructs in order to generate high level producer cell lines using recombinase-assisted recombination events (Karreman et al (1996) NAR 24:1616-1624). A similar system has been developed using the Cre recombinase/IoxP recognition sites of bacteriophage P1 (see PCT/GB00/03837; Vanin et al (1997) J. Virol 71:7820-7826). This was configured into a lentiviral genome such that high titre lentiviral producer cell lines were generated.

By using producer/packaging cell lines, it is possible to propagate and isolate quantities of retroviral vector particles (e.g. to prepare suitable titres of the retroviral vector particles) for subsequent transduction of, for example, a site of interest (such as adult brain tissue). Producer cell lines are usually better for large scale production or vector particles.

Transient transfection has numerous advantages over the packaging cell method. In this regard, transient transfection avoids the longer time required to generate stable vector-producing cell lines and is used if the vector genome or retroviral packaging components are toxic to cells. If the vector genome encodes toxic genes or genes that interfere with the replication of the host cell, such as inhibitors of the cell cycle or genes that induce apoptosis, it may be difficult to generate stable vector-producing cell lines, but transient transfection can be used to produce the vector before the cells die. Also, cell lines have been developed using transient infection that produce vector titre levels that are comparable to the levels obtained from stable vector-producing cell lines (Pear et al 1993, PNAS 90:8392-8396).

Producer cells/packaging cells can be of any suitable cell type. Producer cells are generally mammalian cells but can be, for example, insect cells.

As used herein, the term “producer cell” or “vector producing cell” refers to a cell which contains all the elements necessary for production of retroviral vector particles.

Preferably, the producer cell is obtainable from a stable producer cell line.

Preferably, the producer cell is obtainable from a derived stable producer cell line.

Preferably, the producer cell is obtainable from a derived producer cell line.

As used herein, the term “derived producer cell line” is a transduced producer cell line which has been screened and selected for high expression of a marker gene. Such cell lines support high level expression from the retroviral genome. The term “derived producer cell line” is used interchangeably with the term “derived stable producer cell line” and the term “stable producer cell line.

Preferably the derived producer cell line includes but is not limited to a retroviral and/or a lentiviral producer cell.

Preferably the derived producer cell line is an HIV or EIAV producer cell line, more preferably an EIAV producer cell line.

Preferably the envelope protein sequences, and nucleocapsid sequences are all stably integrated in the producer and/or packaging cell. However, one or more of these sequences could also exist in episomal form and gene expression could occur from the episome.

As used herein, the term “packaging cell” refers to a cell which contains those elements necessary for production of infectious recombinant virus which are lacking in the RNA genome. Typically, such packaging cells contain one or more producer plasmids which are capable of expressing viral structural proteins (such as gag-pol and env, which may be codon optimised) but they do not contain a packaging signal.

The term “packaging signal” which is referred to interchangeably as “packaging sequence” or “psi” is used in reference to the non-coding, cis-acting sequence required for encapsidation of retroviral RNA strands during viral particle formation. In HIV-1, this sequence has been mapped to loci extending from upstream of the major splice donor site (SD) to at least the gag start codon.

Packaging cell lines may be readily prepared (see also WO 92/05266), and utilised to create producer cell lines for the production of retroviral vector particles. As already mentioned, a summary of the available packaging lines is presented in “Retroviruses” (as above).

Also as discussed above, simple packaging cell lines, comprising a provirus in which the packaging signal has been deleted, have been found to lead to the rapid production of undesirable replication competent viruses through recombination. In order to improve safety, second generation cell lines have been produced wherein the 3′ LTR of the provirus is deleted. In such cells, two recombinations would be necessary to produce a wild type virus. A further improvement involves the introduction of the gag-pol genes and the env gene on separate constructs so-called third generation packaging cell lines. These constructs are introduced sequentially to prevent recombination during transfection.

Preferably, the packaging cell lines are second generation packaging cell lines.

Preferably, the packaging cell lines are third generation packaging cell lines.

In these split-construct, third generation cell lines, a further reduction in recombination may be achieved by changing the codons. This technique, based on the redundancy of the genetic code, aims to reduce homology between the separate constructs, for example between the regions of overlap in the gag-pol and env open reading frames.

The packaging cell lines are useful for providing the gene products necessary to encapsidate and provide a membrane protein for a high titre vector particle production. The packaging cell may be a cell cultured in vitro such as a tissue culture cell line. Suitable cell lines include but are not limited to mammalian cells such as murine fibroblast derived cell lines or human cell lines. Preferably the packaging cell line is a human cell line, such as for example: HEK293, 293-T, TE671, HT1080.

Alternatively, the packaging cell may be a cell derived from the individual to be treated such as a monocyte, macrophage, blood cell or fibroblast. The cell may be isolated from an individual and the packaging and vector components administered ex vivo followed by re-administration of the autologous packaging cells.

It is highly desirable to use high-titre virus preparations in both experimental and practical applications. Techniques for increasing viral titre include using a psi plus packaging signal as discussed above and concentration of viral stocks.

As used herein, the term “high titre” means an effective amount of a retroviral vector or particle which is capable of transducing a target site such as a cell.

As used herein, the term “effective amount” means an amount of a regulated retroviral or lentiviral vector or vector particle which is sufficient to induce expression of the NOIs at a target site.

A high-titre viral preparation for a producer/packaging cell is usually of the order of 10⁵ to 10⁷ t.u. per ml. (The titer is expressed in transducing units per ml (t.u./ml) as titred on a standard D17 cell line). For transduction in tissues such as the brain, it is necessary to use very small volumes, so the viral preparation is concentrated by ultracentrifugation. The resulting preparation should have at least 10⁸ t.u./ml, preferably from 10⁸ to 10⁹ t.u./ml, more preferably at least 10⁹ t.u./ml.

The expression products encoded by the NOIs may be proteins which are secreted from the cell. Alternatively the NOI expression products are not secreted and are active within the cell. For some applications, it is preferred for the NOI expression product to demonstrate a bystander effect or a distant bystander effect; that is the production of the expression product in one cell leading to the modulation of additional, related cells, either neighbouring or distant (e.g. metastatic), which possess a common phenotype.

The presence of a sequence termed the central polypurine tract (cPPT) may improve the efficiency of gene delivery to non-dividing cells (see WO 00/31200). This cis-acting element is located, for example, in the EIAV polymerase coding region element. Preferably the genome of the vector system used in the present invention comprises a cPPT sequence.

In addition, or in the alternative, the viral genome may comprise a post-translational regulatory element and/or a translational enhancer.

The NOIs may be operatively linked to one or more promoter/enhancer elements. Transcription of one or more NOI may be under the control of viral LTRs or alternatively promoter-enhancer elements can be engineered in with the transgene. Preferably the promoter is a strong promoter such as CMV. The promoter may be a regulated promoter. The promoter may be tissue-specific. In a preferred embodiment the promoter is glial cell-specific. In another preferred embodiment the promoter is neuron-specific.

Minimal Systems

It has been demonstrated that a primate lentivirus minimal system can be constructed which requires none of the HIV/SIV additional genes vif, vpr, vpx, vpu, tat, rev and nef for either vector production or for transduction of dividing and non-dividing cells. It has also been demonstrated that an EIAV minimal vector system can be constructed which does not require S2 for either vector production or for transduction of dividing and non-dividing cells. The deletion of additional genes is highly advantageous. Firstly, it permits vectors to be produced without the genes associated with disease in lentiviral (e.g. HIV) infections. In particular, tat is associated with disease. Secondly, the deletion of additional genes permits the vector to package more heterologous DNA. Thirdly, genes whose function is unknown, such as S2, may be omitted, thus reducing the risk of causing undesired effects. Examples of minimal lentiviral vectors are disclosed in WO-A-99/32646 and in WO-A-98/17815.

Thus, preferably, the delivery system used in the invention is devoid of at least tat and S2 (if it is an EIAV vector system), and possibly also vif, vpr, vpx, vpu and net More preferably, the systems of the present invention are also devoid of rev. Rev was previously thought to be essential in some retroviral genomes for efficient virus production. For example, in the case of HIV, it was thought that rev and RRE sequence should be included. However, it has been found that the requirement for rev and RRE can be reduced or eliminated by codon optimisation (see below) or by replacement with other functional equivalent systems such as the MPMV system. As expression of the codon optimised gag-pol is REV independent, RRE can be removed from the gag-pol expression cassette, thus removing any potential for recombination with any RRE contained on the vector genome.

In a preferred embodiment the viral genome of the first aspect of the invention lacks the Rev response element (RRE).

In a preferred embodiment, the system used in the present invention is based on a so-called “minimal” system in which some or all of the additional genes have be removed.

Codon Optimisation

Codon optimisation has previously been described in WO99/41397. Different cells differ it their usage of particular codons. This codon bias corresponds to a bias in the relative abundance of particular tRNAs in the cell type. By altering the codons in the sequence so that they are tailored to match with the relative abundance of corresponding tRNAs, It is possible to increase expression. By the same token, it is possible to decrease expression by deliberately choosing codons for which the corrsponding tRNAs are known to be rare in the particular cell type. Thus, an additional degree of translational control is available.

Many viruses, including HIV and other lentiviruses, use a large number of rare codons and by changing these to correspond to commonly used mammalian codons, increased expression of the packaging components in mammalian producer cells can be achieved. Codon usage tables are known in the art for mammalian cells, as well as for a variety of other organisms.

Codon optimisation has a number of other advantages. By virtue of alterations in their sequences, the nucleotide sequences encoding the packaging components of the viral particles required for assembly of viral particles in the producer cells/packaging cells have RNA instability sequences (INS) eliminated from them. At the same time, the amino acid sequence coding sequence for the packaging components is retained so that the viral components encoded by the sequences remain the same, or at least sufficiently similar that the function of the packaging components is not compromised. Codon optimisation also overcomes the Rev/RRE requirement for export, rendering optimised sequences Rev independent. Codon optimisation also reduces homologous recombination between different constructs within the vector system (for example between the regions of overlap in the gag-pol and env open reading frames). The overall effect of codon optimisation is therefore a notable increase in viral titre and improved safety.

In one embodiment only codons relating to INS are codon optimised. However, in a much more preferred and practical embodiment, the sequences are codon optimised in their entirety, with the exception of the sequence encompassing the frameshift site.

The gag-pol gene comprises two overlapping reading frames encoding the gag-pol proteins. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the gag-pol gene. For HIV, the region of overlap extends from nucleotide 1222 downstream of the beginning of gag (wherein nucleotide 1 is the A of the gag ATG) to the end of gag (nt 1503). Consequently, a 281 bp fragment spanning the frameshift site and the overlapping region of the two reading frames is preferably not codon optimised. Retaining this fragment will enable more efficient expression of the gag-pol proteins.

For EIAV the beginning of the overlap has been taken to be nt 1262 (where nucleotide 1 is the A of the gag ATG). The end of the overlap is at 1461 bp. In order to ensure that the frameshift site and the gag-pol overlap are preserved, the wild type sequence has been retained from nt 1156 to 1465.

Derivations from optimal codon usage may be made, for example, in order to accommodate convenient restriction sites, and conservative amino acid changes may be introduced into the gag-pol proteins.

In a highly preferred embodiment, codon optimisation was based on lightly expressed mammalian genes. The third and sometimes the second and third base may be changed.

Due to the degenerate nature of the Genetic Code, it will be appreciated that numerous gag-pol sequences can be achieved by a skilled worker. Also there are many retroviral variants described which can be used as a starting point for generating a codon optimised gag-pol sequence. Lentiviral genomes can be quite variable. For example there are many quasi-species of HIV-1 which are still functional. This is also the case for EIAV. These variants may be used to enhance particular parts of the transduction process. Examples of HIV-1 variants may be found at http://hiv-web.lanl.gov. Details of EIAV clones may be found at the NCBI database: http://www.ncbi.nlm.nih.gov.

The strategy for codon optimised gag-pol sequences can be used in relation to any retrovirus. This would apply to all lentiviruses, including EIAV, FIV, BIV, CAEV, VMR, SIV, HIV-1 and HIV-2. In addition this method could be used to increase expression of genes from HTLV-1, HTLV-2, HFV, HSRV and human endogenous retroviruses (HERV), MLV and other retroviruses.

Codon optimisation can render gag-pol expression Rev independent. In order to enable the use of anti-rev or RRE factors in the retroviral vector, however, it would be necessary to render the viral vector generation system totally Rev/RRE independent. Thus, the genome also needs to be modified. This is achieved by optimising vector genome components. Advantageously, these modifications also lead to the production of a safer system absent of all additional proteins both in the producer and in the transduced cell.

As described above, the packaging components for a retroviral vector include expression products of gag, pol and env genes. In addition, efficient packaging depends on a short sequence of 4 stem loops followed by a partial sequence from gag and env (the “packaging signal”). Thus, inclusion of a deleted gag sequence in the retroviral vector genome (in addition to the full gag sequence on the packaging construct) will optimise vector titre. To date efficient packaging has been reported to require from 255 to 360 nucleotides of gag in vectors that still retain env sequences, or about 40 nucleotides of gag in a particular combination of splice donor mutation, gag and env deletions. It has surprisingly been found that a deletion of all but the N-termnial 360 or so nucleotides in gag leads to an increase in vector titre. Thus, preferably, the retroviral vector genome includes a gag sequence which comprises one or more deletions, more preferably the gag sequence comprises about 360 nucleotides derivable from the N-terminus.

Pseudotyping

In the design of retroviral vector systems it is desirable to engineer particles with different target cell specificities to the native virus, to enable the delivery of genetic material to an expanded or altered range of cell types. One manner in which to achieve this is by engineering the virus envelope protein to alter its specificity. Another approach is to introduce a heterologous envelope protein into the vector particle to replace or add to the native envelope protein of the virus.

The term pseudotyping means incorporating in at least a part of, or substituting a part of, or replacing all of, an env gene of a viral genome with a heterologous env gene, for example an env gene from another virus. Pseudotyping is not a new phenomenon and examples may be found in WO 99/61639, WO-A-98/05759, WO-A-98/05754, WO-A-97/17457, WOA-96/09400, WO-A-91/00047 and Mebatsion et al 1997 Cell 90, 841-847.

Pseudotyping can improve retroviral vector stability and transduction efficiency. A pseudotype of murine leukemia virus packaged with lymphocytic choriomeningitis virus (LCMV) has been described (Miletic et al (1999) J. Virol. 73:6114-6116) and shown to be stable during ultracentrifugation and capable of infecting several cell lines from different species.

In the present invention the vector system may be pseudotyped with at least a part of a rabies G protein or a mutant, variant, homologue or fragment thereof.

Thus, the retroviral delivery system used in the first aspect of the invention comprises a first nucleotide sequence coding for at least a part of an envelope protein; and one or more other nucleotide sequences derivable from a retrovirus that ensure transduction by the retroviral delivery system; wherein the first nucleotide sequence is heterologous with respect to at least one of the other nucleotide sequences; and wherein the first nucleotide sequence codes for at least a part of a rabies G protein or a mutant, variant, homologue or fragment thereof.

There is thus provided the use of a retroviral delivery system comprising a heterologous env region, wherein the heterologous env region comprises at least a part of a rabies G protein or a mutant, variant, homologue or fragment thereof.

The heterologous env region may be encoded by a gene which is present on a producer plasmid. The producer plasmid may be present as part of a kit for the production of retroviral vector particles suitable for use in the first aspect of the invention.

Rabies G Protein

In the present invention the vector system may be pseudotyped with at least a part of a rabies G protein or a mutant, variant, homologue or fragment thereof.

Teachings on the rabies G protein, as well as mutants thereof, may be found in in WO 99/61639 and well as Rose et al., 1982 J. Virol. 43: 361-364, Hanham et al., 1993 J. Virol.,67, 530-542, Tuffereau et al.,1998 J. Virol., 72, 1085-1091, Kucera et al., 1985 J. Virol 55, 158-162, Dietzschold et al., 1983 PNAS 80, 70-74, Seif et al., 1985 J. Virol., 53, 926-934, Coulon et al.,1998 J. Virol., 72, 273-278, Tuffereau et al.,1998 J. Virol., 72, 1085-10910, Burger et al., 1991 J. Gen. Virol. 72. 359-367, Gaudin et al 1995 J Virol 69, 5528-5534, Benmansour et al 1991 J Virol 65, 4198-4203, Luo et al 1998 Microbiol Immunol 42, 187-193, Coll 1997 Arch Virol 142, 2089-2097, Luo et al 1997 Virus Res 51, 3541, Luo et al 1998 Microbiol Immunol 42, 187-193, Coll 1995 Arch Virol 140, 827-851, Tuchiya et al 1992 Virus Res 25, 1-13, Morimoto et al 1992 Virology 189, 203-216, Gaudin et al 1992 Virology 187, 627-632, Whitt et al 1991 Virology 185, 681-688, Dietzschold et al 1978 J Gen Virol 40, 131-139, Dietzschold et al 1978 Dev Biol Stand 40, 45-55, Dietzschold et al 1977 J Virol 23, 286-293, and Otvos et al 1994 Biochim Biophys Acta 1224, 68-76. A rabies G protein is also described in EP-A-0445625.

The present invention provides a rabies G protein having the amino acid sequence shown in SEQ ID NO.3. The present invention also provides a nucleotide sequence capable of encoding such a rabies G protein. Preferably the nucleotide sequence comprises the sequence shown in SEQ ID NO. 4.

These sequences differ from the Genbank sequence as shown below:

| | | | | | | | | |
|---|---|---|---|---|---|---|---|---|
| | v I | Y | T | I | L | D | K | L |
| Genbank sequence | ATT | TAC | ACG | ATA | CTA | GAC | AAG | CTT |
| | I | Y | T | I | P | D | K | L |
| Present Invention | ATT | TAC | ACG | ATC | CCA | GAC | AAG | CTT |

In a preferred embodiment, the vector system of the present invention is or comprises at least a part of a rabies G protein protein having the amino acid sequence shown in SEQ ID NO.3.

The use of rabies G protein provides vectors which, in vivo, preferentially transduce targeted cells which rabies virus preferentially infects. This includes in particular neuronal target cells in vivo. For a neuron-targeted vector, rabies G from a pathogenic strain of rabies such as ERA may be particularly effective. On the other hand rabies G protein confers a wider target cell range in vitro including nearly all mammalian and avian cell types tested (Seganti et al., 1990 Arch Virol. 34,155-163; Fields et al., 1996 Fields Virology, Third Edition, vol.2, Lippincott-Raven Publishers, Philadelphia, N.Y.).

The tropism of the pseudotyped vector particles may be modified by the use of a mutant rabies G which is modified in the extracellular domain. Rabies G protein has the advantage of being mutatable to restrict target cell range. The uptake of rabies virus by target cells in vivo is thought to be mediated by the acetylcholine receptor (AchR) but there may be other receptors to which in binds in vivo (Hanham et al., 1993 J. Virol., 67, 530-542; Tuffereau et al., 1998 J. Virol., 72, 1085-1091). It is thought that multiple receptors are used in the nervous system for viral entry, including NCAM (Thoulouze et al (1998) J. Virol 72(9):7181-90) and p75 Neurotrophin receptor (Tuffereau C et al (1998) Embo J 17(24) 7250-9).

The effects of mutations in antigenic site III of the rabies G protein on virus tropism have been investigated, this region is not thought to be involved in the binding of the virus to the acetylcholine receptor (Kucera et al., 1985 J. Virol 55, 158-162; Dietzschold et al., 1983 Proc Natl Acad Sci 80, 70-74; Seif et al., 1985 J. Virol., 53, 926-934; Coulon et al., 1998 J. Virol., 72, 273-278; Tuffereau et al., 1998 J. Virol., 72, 1085-10910). For example a mutation of the arginine at amino acid 333 in the mature protein to glutamine can be used to restrict viral entry to olfactory and peripheral neurons in vivo while reducing propagation to the central nervous system. These viruses were able to penetrate motor neurons and sensory neurons as efficiently as the wild type, virus, yet transneuronal transfer did not occur (Coulon et al., 1989, J. Virol. 63, 3550-3554). Viruses in which amino acid 330 has been mutated are further attenuated, being unable to infect either motor neurons or sensory neurons after intra-muscular injection (Coulon et al., 1998 J. Virol., 72, 273-278).

Alternatively or additionally, rabies G proteins from laboratory passaged strains of rabies may be used. These can be screened for alterations in tropism. Such strains include the following:

| Genbank accession number | Rabies Strain |
|---|---|
| J02293 | ERA |
| U52947 | COSRV |
| U27214 | NY 516 |
| U27215 | NY 771 |
| U27216 | FLA 125 |
| U52946 | SHBRV |
| M32751 | HEP-Flury |

By way of example, the ERA strain is a pathogenic strain of rabies and the rabies G protein from this strain can be used for transduction of neuronal cells. The sequence of rabies G from the ERA strains is in the GenBank database (accession number J02293). This protein has a signal peptide of 19 amino acids and the mature protein begins at the lysine residue 20 amino acids from the translation initiation methionine. The HEP-Flury strain contains the mutation from arginine to glutamine at amino acid position 333 in the mature protein which correlates with reduced pathogenicity and which can be used to restrict the tropism of the viral envelope. WO 99/61639 discloses the nucleic and amino acid sequences for a rabies virus strain ERA (Genbank locus RAVGPLS, accession M38452).

Mutants, Variants, Homologues and Fragments

The vector system is or comprises at least part of a wild-type rabies G protein or a mutant, variant, homologue or fragment thereof.

The term “wild type” is used to mean a polypeptide having a primary amino acid sequence which is identical with the native protein (i.e., the viral protein).

The term “mutant” is used to mean a polypeptide having a primary amino acid sequence which differs from the wild type sequence by one or more amino acid additions, substitutions or deletions. A mutant may arise naturally, or may be created artificially (for example by site-directed mutagenesis). Preferably the mutant has at least 90% sequence identity with the wild type sequence. Preferably the mutant has 20 mutations or less over the whole wild-type sequence. More preferably the mutant has 10 mutations or less, most preferably 5 mutations or less over the whole wild-type sequence.

The term “variant” is used to mean a naturally occurring polypeptide which differs from a wild-type sequence. A variant may be found within the same viral strain (i.e. if there is more than one isoform of the protein) or may be found within a different strains. Preferably the variant has at least 90% sequence identity with the wild type sequence. Preferably the variant has 20 mutations or less over the whole wild-type sequence. More preferably the variant has 10 mutations or less, most preferably 5 mutations or less over the whole wild-type sequence.

Here, the term “homologue” means an entity having a certain homology with the wild type amino acid sequence and the wild type nucleotide sequence. Here, the term “homology” can be equated with “identity”.

In the present context, an homologous sequence is taken to include an amino acid sequence which may be at least 75, 85 or 90% identical, preferably at least 95 or 98% identical to the subject sequence. Typically, the homologues will comprise the same active sites etc; as the subject amino acid sequence. Although homology can also be considered in terms of similarity (i.e. amino acid residues having similar chemical properties/functions), in the context of the present invention it is preferred to express homology in terms of sequence identity.

In the present context, an homologous sequence is taken to include a nucleotide sequence which may be at least 75, 85 or 90% identical, preferably at least 95 or 98% identical to the subject sequence. Typically, the homologues will comprise the same sequences that code for the active sites etc. as the subject sequence. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and 4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However, for some applications, it is preferred to use the GCG Bestfit program. A new tool, called BLAST 2 Sequences is also available for comparing protein and nucleotide sequence (see FEMS Microbiol Lett 1999 174(2): 247-50; FEMS Microbiol Lett 1999 177(1): 187-8 and tatiana@ncbi.nim.nih.gov).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

The sequences may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

The present invention also encompasses homologous substitution (substitution and replacement are both used herein to mean the interchange of an existing amino acid residue, with an alternative residue) may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid omithine (hereinafter referred to as B), norleucine omithine (hereinafter referred to as 0), pyriylalanine, thienylalanine, naphthylalanine and phenylglycine.

Replacements may also be made by unnatural amino acids include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, β-alanine*, L-α-amino butyric acid*, L-γ-amino butyric acid*, L-α-amino isobutyric acid*, L-α-amino caproic acid#, 7-amino heptanoic acid*, L-methionine sulfone#, L-norleucine*, L-norvaline*, p-nitro-L-phenylalanine*, L-hydroxyprolin#, L-thioproline*, methyl derivatives of phenylalanine (Phe) such as 4-methyl-Phe*, pentamethyl-Phe*, L-Phe (4-amino)#, L-Tyr (methyl)*, L-Phe (4-isopropyl)*, L-Tic (1,2,3,4-tetrahydroisoquinolin-3-carboxyl acid)*, L-diaminopropionic acid™ and L-Phe (4-benzyl)*. The notation * has been utilised for the purpose of the discussion above (relating to homologous or non-homologous substitution), to indicate the hydrophobic nature of the derivative whereas #* has been utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell DC, Trends Biotechnol. (1995) 13(4), 132-134.

The term “fragment” indicates that the polypeptide comprises a fraction of the wild-type amino acid sequence. It may comprise one or more large contiguous sections of sequence or a plurality of small sections. The polypeptide may also comprise other elements of sequence, for example, it may be a fusion protein with another protein. Preferably the polypeptide comprises at least 50%, more preferably at least 65%, most preferably at least 80% of the wild-type sequence.

With respect to function, the mutant, variant, homologue or fragment should be capable of transducing TH positive neurones when used to pseudotype an appropriate vector.

The mutant, variant, homologue or fragment rabies G sequence should alternatively or in addition, be capable of conferring the capacity for retrograde transport on the vector system.

The vector delivery system used in the present invention may comprise nucleotide sequences that can hybridise to the nucleotide sequence presented herein (including complementary sequences of those presented herein). In a preferred aspect, the present invention covers nucleotide sequences that can hybridise to the nucleotide sequence of the present invention under stringent conditions (e.g. 65° C. and 0.1 SSC) to the nucleotide sequence presented herein (including complementary sequences of those presented herein).

A potential advantage of using the rabies glycoprotein is the detailed knowledge of its toxicity to man and other animals due to the extensive use of rabies vaccines. In particular phase 1 clinical trials have been reported on the use of rabies glycoprotein expressed from a canarypox recombinant virus as a human vaccine (Fries et al., 1996 Vaccine 14, 428-434), these studies concluded that the vaccine was safe for use in humans.

Another advantage of using a rabies G pseudotyped vector system to transduce a TH positive neuron is that it is capable of retrograde transport (see below).

Both rabies G and VSV-G pseudotyped vector systems have been shown to be capable of transducing TH positive neurons.

TH Positive Neurons

As used herein, the term “TH positive neurons” are neural cells which are capable of producing tyrosine hydroxylase (TH). The production of tyrosine hydroxylase can be determined by known techniques which measure production of tyrosine hydroxylase mRNA (polymerase chain reaction (PCR), Northern blotting) or protein (immunolabelling, radiolabelling, ELISA-based techniques). Also, the production of metabolites may be measured by known techniques including HPLC with electrochemical detection. TH is expressed by dopaminergic neurons, noradrenergic neurons and adrenal cells.

Mesencephalic, catecholaminergic TH positive cells are capable of producing dopamine. The production of dopamine and noradrenaline is summarised below:

Tyrosine-1→L-DOPA-2→Dopamine-3→noradrenaline

1=Tyrosine hydroxylase

2=DOPA decarboxylase

3=Dopamine-betahydroxylase

Noradrenaergic neurones express all three enzymes, whereas dopaminergic neurones express Tyrosine hydroxylase and DOPA decarboxylase, but lack Dopamine-betahydroxylase.

Tyrosine hydroxylase is the rate-limiting enzyme in the biochemical pathway for dopamine production and is commonly used in the art as a marker for dopaminergic neurons. Dopaminergic neurons may be distinguished from noradrenergic neurones by the absence of Dopamine betahydroxylase within the cells.

TH positive cells may be found in or isolated from dopaminergic neural tissue. Dopaminergic neural tissue is derivable from regions of the CNS which, in the mature state, contains significant numbers of dopaminergic cell bodies. Dopaminergic neural tissue is found in regions of the retina, olfactory bulb, hypothalamus, dorsal motor nucleus, nucleus tractus solitarious, periaqueductal gray matter, ventral tegmenum, and substantia nigra.

EOIs/NOIs

In a broad aspect, the present invention relates to a vector system that is capable of transporting an entity of interest (“EOI”).

The EOI may be a chemical compound, a biological compound or combinations thereof. By way of example, the EOI may be a protein (such as a growth factor), a nucleotide sequence, an organic and/or an inorganic pharmaceutical (such as an analgesic, an anti-inflammatory, a hormone, a lipid), or combinations thereof.

Preferably the EOI is one or more NOIs (nucleotide sequences of interest)—wherein said NOIs may be delivered to a target cell in vivo or in vitro.

If the vector system of the present invention is a viral vector system, then it is possible to manipulate the viral genome so that viral genes are replaced or supplemented with one or more NOIs which may be heterologous NOIs.

The term “heterologous” refers to a nucleic acid or protein sequence linked to a nucleic acid or protein sequence to which it is not naturally linked.

In the present invention, the term NOI includes any suitable nucleotide sequence, which need not necessarily be a complete naturally occurring DNA or RNA sequence. Thus, the NOI can be, for example, a synthetic RNA/DNA sequence, a recombinant RNA/DNA sequence (i.e. prepared by use of recombinant DNA techniques), a cDNA sequence or a partial genomic DNA sequence, including combinations thereof. The sequence need not be a coding region. If it is a coding region, it need not be an entire coding region. In addition, the RNAIDNA sequence can be in a sense orientation or in an anti-sense orientation. Preferably, it is in a sense orientation. Preferably, the sequence is, comprises, or is transcribed from cDNA.

The retroviral vector genome may generally comprise LTRs at the 5′ and 3′ ends, suitable insertion sites for inserting one or more NOI(s), and/or a packaging signal to enable the genome to be packaged into a vector particle in a producer cell. There may even be suitable primer binding sites and integration sites to allow reverse transcription of the vector RNA to DNA, and integration of the proviral DNA into the target cell genome. In a preferred embodiment, the retroviral vector particle has a reverse transcription system (compatible reverse transcription and primer binding sites) and an integration system (compatible integrase and integration sites).

The EOI/NOI may be or-encode a protein of interest (“POI”). In this way, the vector delivery system could be used to examine the effect of expression of a foreign gene on the target cell (such as a TH positive neuron). For example, the retroviral delivery system could be used to screen a cDNA library-for a particular effect on a TH positive neuron.

For example, one could identify new survival/neuroprotective factors for dopaminergic neurons, which would enable transfected TH+ cells to persist in the presence of an apoptosis-inducing factor.

The EOI/NOI may be capable of integrating in the genome of a target cell.

The EOI/NOI may be capable of blocking or inhibiting the expression of a gene in the target cell (which may be a TH-positive neuron). For example, the NOI may be an antisense sequence. The inhibition of gene expression using antisense technology is well known.

The EOI/NOI or a sequence derived from the NOI may be capable of “knocking out” the expression of a particular gene in the target cell (for example, a TH positive neuron). There are several “knock out” strategies known in the art. For example, the NOI may be capable of integrating in the genome of the TH positive neuron so as to disrupt expression of the particular gene. The NOI may disrupt expression by, for example, introducing a premature stop codon, by rendering the downstream coding sequence out of frame, or by affecting the capacity of the encoded protein to fold (thereby affecting its function).

Alternatively, the EOI/NOI may be capable of enhancing or inducing ectopic expression of a gene in the target cell (which may be a TH+ neuron). The NOI or a sequence derived therefrom may be capable of “knocking in” the expression of a particular gene.

Transduced TH positive neurones which express a particular gene, or which lack the expression of a particular gene have applications in drug discovery and target validation. The expression system could be used to determine which genes have a desirable effect on TH positive neurones, such as those genes or proteins which are able to prevent or reverse the triggering of apoptosis in the cells. Equally, if the inhibition or blocking of expression of a particular gene is found to have an undesirable effect on the TH positive neuron, this may open up possible therapeutic strategies which ensure that expression of the gene is not lost.

An EOI/NOI delivered by the vector delivery system may be capable of immortalising the target cell. A number of immortalisation techniques are known in the art (see for example Katakura Y et al (1998) Methods Cell Biol. 57:69-91).

The term “immortalised” is used herein to cells capable of growing in culture for greater than 10 passages, which may be maintained in continuous culture for greater than about 2 months.

Immortalised TH positive neurones are useful in experimental procedures, screening programmes and in therapeutic applications. For example, immortalised TH+ neurones may be used for transplantation, in particular to treat Parkinson's disease.

An EOI/NOI delivered by the vector delivery system may be used for selection or marker purposes. For example, the NOI may be a selection gene, or a marker gene. Many different selectable markers have been used successfully in retroviral vectors. These are reviewed in “Retroviruses” (1997 Cold Spring Harbour Laboratory Press Eds: JM Coffin, SM Hughes, HE Varmus pp 444) and include, but are not limited to, the bacterial neomycin and hygromycin phosphotransferase genes which confer resistance to G418 and hygromycin respectively; a mutant mouse dihydrofolate reductase gene which confers resistance to methotrexate; the bacterial gpt gene which allows cells to grow in medium containing mycophenolic acid, xanthine and aminopterin; the bacterial hisD gene which allows cells to grow in medium without histidine but containing histidinol; the multidrug resistance gene (mdr) which confers resistance to a variety of drugs; and the bacterial genes which confer resistance to puromycin or phleomycin. All of these markers are dominant selectable and allow chemical selection of most cells expressing these genes.

The EOI may have or encode a protein which has a therapeutic effect. For example, an NOI delivered by the vector delivery system may be a therapeutic gene—in the sense that the gene itself may be capable of eliciting a therapeutic effect or it may code for a product that is capable of eliciting a therapeutic effect.

In one preferred embodiment, the EOI is (or the NOI is capable of encoding) a neuroprotective molecule. In particular, the EOI(s) may be (or the NOI(s) may encode) molecules which prevent TH-positive neurons from dying or which stimulate regeneration and functional recovery in the damaged nigrostriatal system. In another preferred embodiment; the EOI is (or the NOI is capable of encoding) an enzyme or enzymes responsible for L-DOPA or dopamine synthesis such as tyrosine hydroxylase.

In accordance with the present invention, suitable EOIs include those that are (or can produce entities) of therapeutic and/or diagnostic application such as, but not limited to: cytokines, chemokines, hormones, antibodies, anti-oxidant molecules, engineered immunoglobulin-like molecules, a single chain antibody, fusion proteins, enzymes, immune co-stimulatory molecules, immunomodulatory molecules, anti-sense RNA, a transdominant negative mutant of a target protein, a toxin, a conditional toxin, an antigen, a tumour suppresser protein and growth factors, membrane proteins, vasoactive proteins and peptides, anti-viral proteins and ribozymes, and derivatives thereof (such as with an associated reporter group). The EOI may be a pro-drug activating enzyme. The EOI may be an NOI which encodes a member of this list.

As used herein, “antibody” includes a whole immunoglobulin molecule or a part thereof or a bioisostere or a mimetic thereof or a derivative thereof or a combination thereof. Examples of a part thereof include: Fab, F(ab)′_{2,} and Fv. Examples of a bioisostere include single chain Fv (ScFv) fragments, chimeric antibodies, bifunctional antibodies.

The term “mimetic” relates to any chemical which may be a peptide, polypeptide, antibody or other organic chemical which has the same binding specificity as the antibody.

The term “derivative” as used herein includes chemical modification of an antibody.

Illustrative of such modifications would be replacement of hydrogen by an alkyl, acyl, or amino group.

The EOI/NOI may also be or encode an antiapoptotic factor or a neuroprotective molecule. The survival of cells during programmed cell death depends critically on their ability to access “trophic” molecular signals derived primarily from interactions with other cells. For example, the NOI may encode a neurotrophic factor, such as ciliary neurotrophic factor (CNTF) or glial cell-derived neurotrophic factor.(GDNF) or it may be a gene involved in control of the cell death cascade (such as Bcl-2). This may be useful in therapeutic strategies involving arresting neuronal and glial cell death induced by injury, disease, and/or aging in humans.

In a further embodiment the present invention provides a method for screening for neuroprotective and/or survival factors for TH positive neurones, which comprises the following steps:

(i) transducing TH-positive neurons with a cDNA library capable of encoding a plurality of candidate compounds;

(ii), exposing the transduced TH-positive neurones to an apoptosis-inducing agent; and

(iii) selecting a candidate compound which enables the TH-positive neuron in which it is expressed to avoid apoptosis during step (ii).

The TH positive cells may be transduced using a system as described in connection with the use of the first aspect of the invention.

The present invention also provides a neuroprotective and/or survival factor for TH positive neurones identified by the above-mentioned method.

The EOI/NOI may be or encode an enzyme involved in dopamine synthesis. For example, the enzyme may be one of the following: Tyrosine Hydroxylase, GTP-cyclohydrolase I and/or Aromatic Amino Acid Dopa Decarboxylase. The sequences of all three genes are available: Accession Nos. X05290, U19523 and M76180 respecively.

Alternatively the EOI/NOI may be or encode the vesicular monoamine transporter 2 (VMAT2). In a preferred embodiment the viral genome comprises an NOI encoding Aromatic Amino Acid Dopa Decarboxylase and an NOI encoding VMAT 2. Such a genome may be used in the treatment of Parkinson's disease, in particular in conjunction with peripheral administration of L-DOPA.

Alternatively the EOI/NOI may be or encode a factor capable of blocking or inhibiting degeneration in the nigrostriatal system. An example of such a factor is a neurotrophic factor. For example the NOI may encode glial cell-line derived neurotrophic factor (GDNF) or brain-derived neurotrophic factor (BDNF).

Particularly useful, for example in the treatment of Parkinson's disease, are multicistronic lentiviral vectors encoding two or more factors. Such a vector may encode Tyrosine Hydroxylase, GTP-cyclohydrolase I and/or Aromatic Amino Acid Dopa Decarboxylase.

If the disease is associated with the death of TH+ neurons, the EOI/NOI may act to prevent TH positive neurons from dying and/or stimulate division of neurons and/or differentiation of neuronal precursors for neuronal regeneration purposes.

If the disease is associated with an impaired function of TH positive neurons, the EOI/NOI may act to restore or replace such a function. For example, if the dopamine producing activity of TH+ neurones was impaired due to the restricted activity of a certain gene, the EOI/NOI may serve to activate or replace the particular gene.

Screening Methods

In further aspect the present invention also provides a number of screening methods, factors isolatable by such methods, and uses for such factors. These aspects of the invention are presented below by way of numbered paragraphs:

1. A method for screening for trophic factors for TH positive neurones, which comprises the following steps:

(i) transducing expressor cells with a cDNA library capable of encoding a plurality of candidate compounds;

(ii) expressing the plurality of candidate compounds and allowing the expressed compounds to come into contact with TH-positive neurones; and

(iii) selecting a candidate compound which causes migration and/or changes in morphology of the TH-positive neurons.

The expresser cells may be TH negative neuronal cells, for example glial cells.

The expressor cells may be transduced using a lentiviral vector system, for example a system as used in the first aspect of the invention.

2. A trophic factor for TH positive neurones identified by the method of paragraph 1.

3. A method for screening for neuroprotective and/or survival factors for TH positive neurones, which comprises the following steps:

(i) transducing TH-positive neurons with a cDNA library capable of encoding a plurality of candidate compounds;

(ii) exposing the transduced TH-positive neurones to an apoptosis-inducing agent; and

(iii) selecting a candidate compound which enables the TH-positive neuron in which it is expressed to avoid apoptosis during step (ii).

The TH positive cells may be transduced using a system as described in connection with the use of the first aspect of the invention.

4. A neuroprotective and/or survival factor for TH positive neurones identified by the method of paragraph 3.

5. A method for screening for differentiation factors capable of stimulating differentiation of neural progenitor cells, which comprises the following steps:

(i) transducing expressor cells with a cDNA library capable of encoding a plurality of candidate compounds;

(ii) expressing the plurality of candidate compounds and allowing the expressed compounds to come into contact with neural progenitor cells;

(iii) selecting a candidate compound which causes differentiation of neural progenitor cells.

The expressor cells may be TH negative neuronal cells, for example glial cells. The expressor cells may be part of a mixture of cells, for example general mesencephalic cells.

The expressor cells may be transduced using a lentiviral vector system, for example a system as used in the first aspect of the invention.

6. A method according to paragraph 5, in which in step (iii) differentiation is monitored by measuring the appearance TH-positive cells.

7. A differentiation factor identified by the method of paragraph 5 or 6.

8. A differentiation factor according to paragraph 7, for use in differentiating a graft of neuroprogenitor cells after transplantation.

9. A method for treating and/or preventing a disease in a subject in need of same, said method comprising the following steps:

(i) transplanting a neuroprogenitor cell into the subject; and

(ii) differentiating the transplanted cell using a differentiation factor according to paragraph 8.

Pharmaceutical Compositions

The present invention also provides the use of a vector delivery system in the manufacture of a pharmaceutical composition. The pharmaceutical composition may be used to deliver an EOI, such as an NOI, to a target cell in need of same. The target cell may, for example be a TH positive neuron.

The vector delivery system can be a non-viral delivery system or a viral delivery system.

In some preferred aspects, the vector delivery system is a viral delivery vector system.

In some further preferred aspects, the vector delivery system is a retroviral vector delivery system.

The pharmaceutical composition may be used for treating an individual by gene therapy, wherein the composition comprises or is capable of producing a therapeutically effective amount of a vector system according to the present invention.

The method and pharmaceutical composition of the invention may be used to treat a human or animal subject. Preferably the subject is a mammalian subject. More preferably the subject is a human. Typically, a physician will determine the actual dosage which will be most suitable for an individual subject and it will vary with the age; weight and-response-of-the particular patient.

The composition may optionally comprise a pharmaceutically acceptable carrier, diluent, excipient or adjuvant. The choice of pharmaceutical carrier, excipient or diluent can be selected with regard to the intended route of administration and standard pharmaceutical practice. The pharmaceutical compositions may comprise as (or in addition to) the carrier, excipient or diluent, any suitable binder(s), lubricant(s), suspending agent(s), coating agent(s), solubilising agent(s), and other carrier agents that may aid or increase the viral entry into the target site (such as for example a lipid delivery system).

Where appropriate, the pharmaceutical compositions can be administered by any one dr more of: inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intracavernosally, intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

The vector system used in the present invention may conveniently be administered by direct injection into the patient For the treatment of neurodegenerative disorders, such as Parkinson's disease, the system may be injected into the brain. The system may be injected directly into any target area of the brain (for example, the striatum or substantia nigra). Alternatively, if a rabies G-pseudotyped vector is used, the system can be injected into a given area, and the target area transduced by retrograde transport of the vector system.

Retrograde Transport

The present invention provides the use of a vector system to transduce a target site, wherein the vector system travels to the site by retrograde transport.

The cell body is where a neuron synthesises new cell products. Two types of transport systems carry materials from the cell body to the axon terminals and back. The slower system, which moves materials 1-5 mm per day is called slow axonal transport. It conveys axoplasm in one direction only (from the cell body toward the axon terminals (anterograde transport)). There is also “Fast transport” which is responsible for the movement of membranous organelles at 50-200 mm per day away from the cell body (anterograde) or back to the cell body (retrograde) (Hirokawa (1997) Curr Opin Neurobiol 7(5):605-614).

Vector systems comprising rabies G protein are capable of retrograde transport (i.e. travelling towards the cell body). The precise mechanism of retrograde transport is unknown, however. It is thought to involve transport of the whole viral particle, possibly in association with an internalised receptor. The fact that vector systems comprising rabies G can be specifically be transported in this manner (as demonstrated herein) suggests that the env protein may be involved.

HSV, adenovirus and hybrid HSV/adeno-associated virus vectors have all been shown to be transported in a retrograde manner in the brain (Horellou and Mallet (1997) Mol Neurobiol 15(2) 241-256; Ridoux et al (1994) Brain Res 648:171-175; Constantini et al (1999) Human Gene Therapy 10:2481-2494). Injection of Adenoviral vector system expressing glial cell line derived neurotrophic factor (GDNF) into rat striatum allows expression in both dopaminergic axon terminals and cell bodies via retrograde transport (Horellou and Mallet (1997) as above; Bilang-Bleuel et al (1997) Proc. Natl. Acd. Sci. USA 94:8818-8823).

Retrograde transport can be detected by a number of mechanisms known in the art. In the present examples, a vector system expressing a heterologous gene is injected into the striatum, and expression of the gene is detected in the substantia nigra. It is clear that retrograde transport along the neurons which extend from the substantia nigra to the basal ganglia is responsible for this phenomenon. It is also known to monitor labelled proteins or viruses and directly monitor their retrograde movement using real time confocal microscopy.(Hirokawa.(1997) as above)., By retrograde transport, it is possible to get expression in both the axon terminals and the cell bodies of transduced neurons. These two parts of the cell may be located in distinct areas of the nervous system. Thus, a single administration (for example, injection) of the vector system of the present invention may transduce many distal sites.

The present invention thus also provides the use of a vector system where the vector system is or comprises at least part of rabies G to transduce a target site, which comprises the step of administration of the vector system to an administration site which is distant from the target site.

The target site may be any site of interest which is anatomically connected to the administration site. The target site should be capable of receiving vector from the administration site by axonal transport, for example anterograde or (more preferably) retrograde transport. For a given administration site, a number of potential target sites may exist which can be identified using retrograde tracers by methods known in the art (Ridoux et al (1994) as above).

For example, intrastriatal injection of HSV/AAV amplicon vectors causes transgene expression in the substantia nigra, cortex, several thalamic nuclei (posterior, paraventricular, parafasicular, reticular), prerubral field, deep mesencephalic nuclei, mesencephalic grey nucleus, and intrastitial nucleus of the medial as well as dorsal longitudinal fasiculus (Constantini et al (1999) as above).

A target site is considered to be “distant from the administration” if it is (or is mainly) located in a different region from the administration site. The two sites may be distinguished by their spatial location, morphology and/or function.

In the brain, the basal ganglia consist of several pairs of nuclei, the two members of each pair being located in opposite cerebral hemispheres. The largest nucleus is the corpus striatum which consists of the caudate nucleus and the lentiform nucleus. Each lentiform nucleus is, in turn, subdivided into a lateral part called the putamen and a medial part called the globus pallidus. The substantia nigra and red nuclei of the midbrain and the subthalamic nuclei of the diencephalon are functionally linked to the basal ganglia. Axons from the substantia nigra terminate in the caudate nucleus or the putamen. The subthalamic nuclei connect with the globus pallidus. For conductivity in basal ganglia of the rat see Oorschot (1996) J. Comp. Neurol. 366:580-599.

In a preferred embodiment, the administration site is the striatum of the brain, in particular the caudate putamen. Injection into the putamen can label target sites located in various distant regions of the brain, for example, the globus pallidus, amygdala, subthalamic nucleus or the substantia nigra. Transduction of cells in the pallidus commonly causes retrograde labelling of cells in the thalamus. In a preferred embodiment the (or one of the) target site(s) is the substantia nigra.

In another preferred embodiment the vector system is injected directly into the spinal cord. This administration site accesses distal connections in the brain stem and cortex

Within a given target site; the vector system may transduce a target cell. The target cell may be a cell found in nervous tissue, such as a neuron, astrocyte, oligodendrocyte, microglia or ependymal cell. In a preferred embodiment, the target cell is a neuron, in particular a TH positive neuron.

The vector system is preferably administered by direct injection. Methods for injection into the brain (in particular the striatum) are well known in the art (Bilang-Bleuel et al (1997) Proc. Acad. Natl. Sci. USA 94:8818-8823; Choi-Lundberg et al (1998) Exp. Neurol.154:261-275; Choi-Lundberg et al (1997) Science 275:838-841; and Mandel et al (1997)) Proc. Acad. Natl. Sci. USA 94:14083-14088). Stereotaxic injections may be given.

As mentioned above, for transduction in tissues such as the brain, it is necessary to use very small volumes, so the viral preparation is concentrated by ultracentrifugation. The resulting preparation should have at least 10⁸ t.u./ml, preferably from 10⁸ to 10¹⁰ t.u./ml, more preferably at least 10⁹ t.u./ml. (The titer is expressed in transducing units per ml (t.u./ml) as titred on a standard D17 cell line). It has been found that improved dispersion of transgene expression can be obtained by increasing the number of injection sites and decreasing the rate of injection (Horellou and Mallet (1997) as above). Usually between 1 and 10 injection sites are used, more commonly between 2 and 6. For a dose comprising 1-5×1.09 t.u./ml, the rate of injection is commonly between 0.1 and 10 μl/min, usually about 1 μl/min.

In another preferred embodiment the vector system is administered-to a peripheral administration site. The vector may be administered to any part of the body from which it can travel to the target site by retrograde transport. In other words the vector may be administered to any part of the body to which a neuron within the target site projects.

The “periphery” can be considered to be all part of the body other than the CNS (brain and spinal cord). In particular, peripheral sites are those which are distant to the CNS. Sensory neurons may be accessed by administration to any tissue which is innervated by the neuron. In particular this includes the skin, muscles and the sciatic nerve.

In a highly preferred embodiment the vector system is administered intramuscularly. In this way, the system can access a distant target site via the neurons which innervate the innoculated muscle. The vector system may thus be used to access the CNS (in particular the spinal cord), obviating the need for direct injection into this tissue. There is thus provided a non-invasive method for transducing a neuron within the CNS. Muscular administration also enables multiple doses to be administered over a prolonged period.

Another advantage with this system is that it is possible to target particular groups of cells (e.g. sets of neurons), or a particular neural tract by choosing a particular administration site.

In a preferred embodiment, the vector system is used to transduce a neuron which innervates (directly or indirectly) the administration site. The target neuron may, for example, be a motoneuron or a sensory neuron.

Sensory neurons may also be accessed by administration to any tissue which is innervated by the neuron. In particular this includes the skin and the sciatic nerve. Where a patient is suffering from pain (in particular slow, chronic pain), the particular sensory neuron(s) involved in transmitting the pain may be targetted by administration of the vector system directly into the area of pain.

Diseases

The vector system used in the present invention is particularly useful in treating and/or preventing a disease which is associated with the death or impaired function of cells of the nervous tissue, such as neurons and/or glial cells.

In particular, the vector system used in the present invention may be used to treat and/or prevent a disease which is associated with the death or impaired function of TH positive neurons.

Diseases which may be treated include, but are not limited to: Parkinson's disease; motor neuron disease and Huntington's disease.

In particular, the vector system used in the present invention is useful in treating and/or preventing Parkinson's disease.

Since the vector system of the present invention may be used for non-invasive access to the CNS, it is suitable for the treatment and/or prevention of any disease which affects the brain and/or spinal cord. The capacity to target motoneurons makes it particulaly suitable for the treatment and/or prevention of motoneuron diseases. For example, Amyotrophic Lateral Sclerosis (ALS) may be treatable with the use of anti-apoptotic factors. Spinal muscular atrophy (in neonates) may be preventable or treatable by replacing survival motor neuron gene 1, in order to avoid apoptosis.

The capacity to target sensory neurons make the system attractive for use in pain relief. There are also potential applications in hyperanalgesia. For example, encephalins may be used to regrow sensory neurons in conditions such as paraplegia. The vector system could be used to provide RORβ2 at the target site.

Transplantation

The present invention also provides a genetically modified (e.g immortalised) TH positive neuron and its use in transplantation methods.

Grafting protocols using embryonic dopamiergic neurons, equivalent cells from other species, and neural progeniter cells are known (reviewed in Dunnett and Bjorklund (1999) Nature Vol 399 Supplement pages A32-39). Similar techniques could be used for grafting the cells of the present invention.

The invention will now be further described by way of Examples, which are meant to serve to assist one of ordinary skill in the art in carrying out the invention and are not intended in any way to limit the scope of the invention. The Examples refer to the Figures. In the Figures:

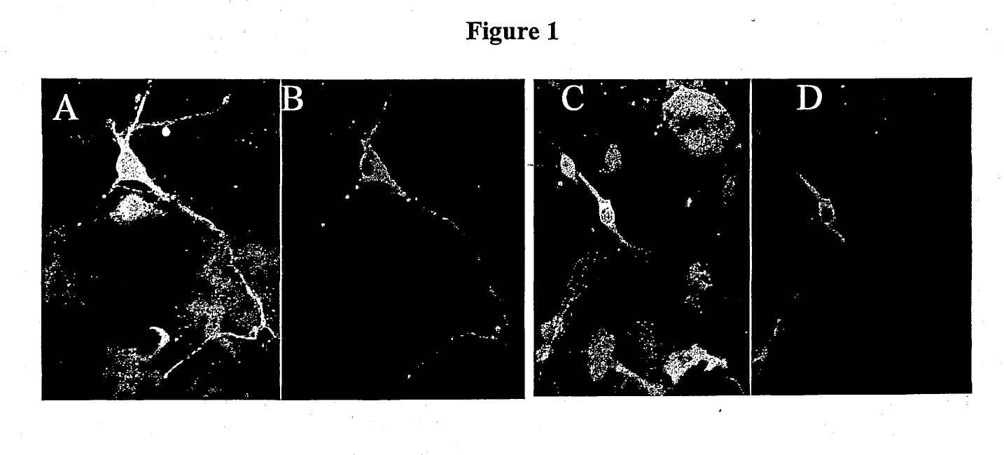

FIG. 1 shows the expression of EIAV (pONY8 GFP) Rabies-G viral vector in TH+ neurons of mouse E14 mesencephalic cultures: (A) Image of GFP+ neuron on top of a layer of transduced astrocytes (flat cells slightly out of focus). (B) Image of same neuron also staining for TH. Transduction for (A,B) is at an MOI of 1. (C) Image of GFP+ neurons on top of astrocytes; (D) Two of these GFP neurons also stain for TH although others are clearly negative. None of the glia stain with TH. Transduction for (C,D) is at an MOI of 10.

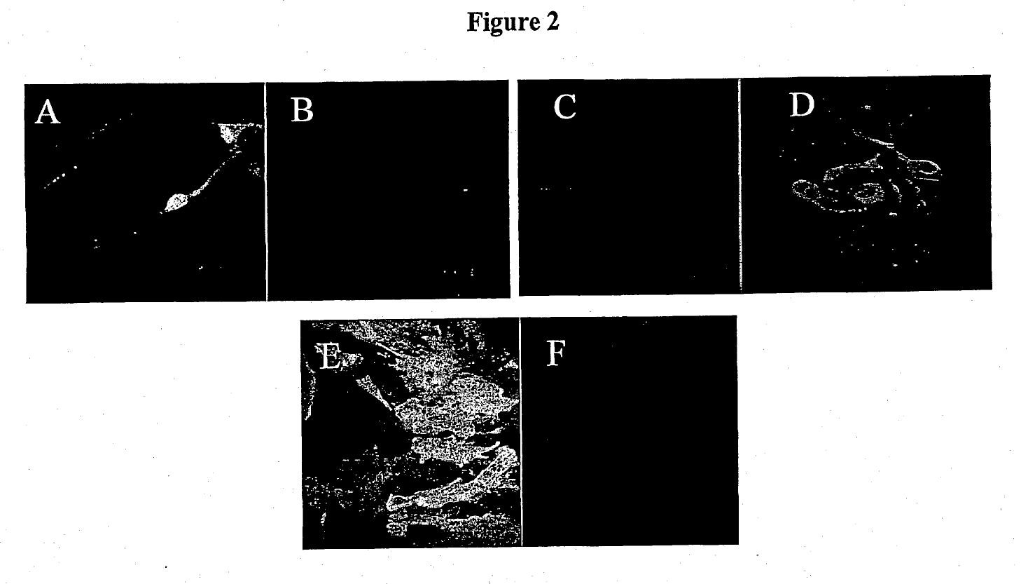

FIG. 2 shows the expression of EIAV (pONY8 GFP) Rabies-G viral vector in glia and TH-neurons in mouse E14 mesencephalic cultures: (A) A field in which several GFP+ neurons could be found that are TH-(B). (C) Control cells treated only with polybrene and no virus expressing TH (D) but not GFP. (E) A clump of GFP+astrocytes which express no TH (F). MOI for these transductions is 1.

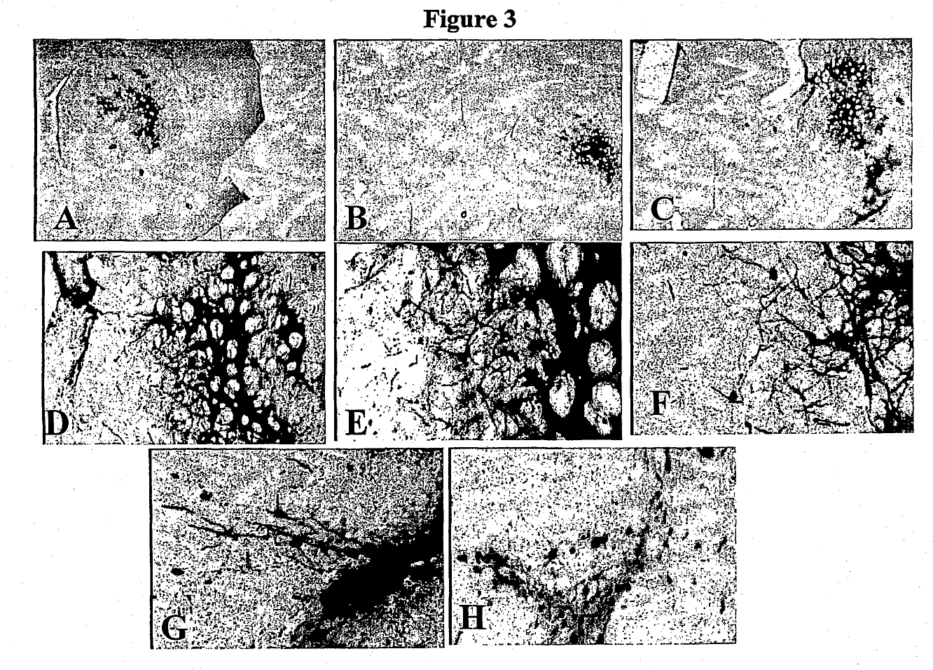

FIG. 3 shows the effect of transduction of the adult rat striatum with EIAV pONY8Z VSVG viral vector (1 week post-injection): Panels A-C correspond to 3 independent 50 μm coronal sections stained with X-gal. An average of fifty of such sections are stained per animal, indicating that the transduction spans the rat striatum. Panels D-H represent higher magnification of the section in C showing that many of the cells transduced have neuronal morphology both within caudate putamen (D-F) and in nucleus accumbens (G-H).

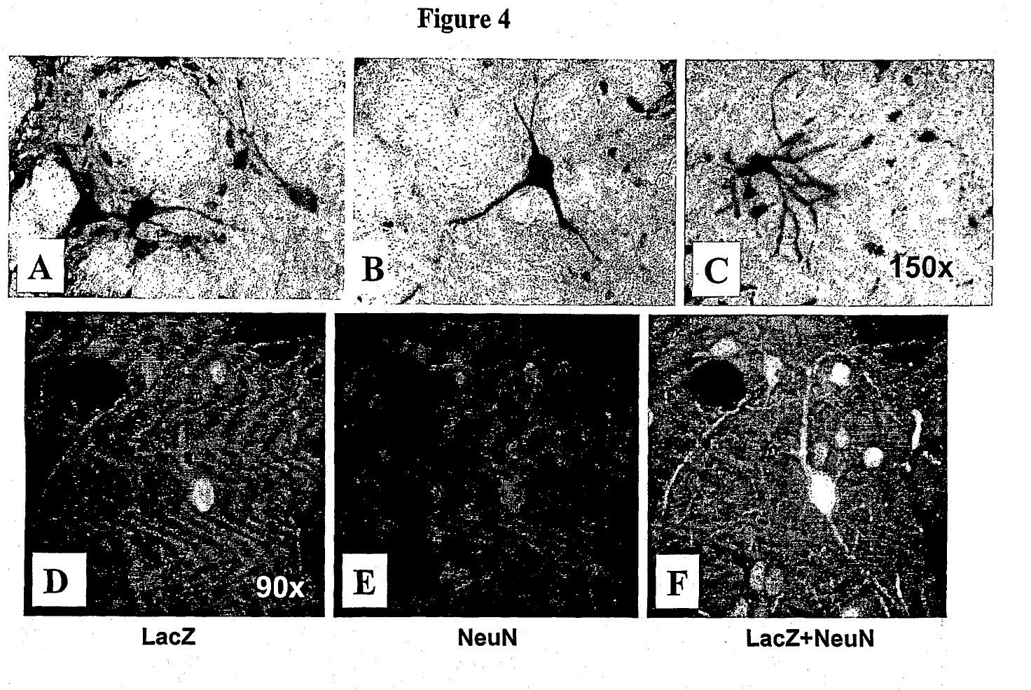

FIG. 4 shows cell types transduced in the adult rat striatum with EIAV pONY8Z VSVG viral vector. High magnification images of striatal neurons: larger aspiny interneurons (A,B) and medium-sized spiny neurons (C) are stained. LacZ expressing cells (D) colocalised with the neuronal postmitotic marker NeuN (E) giving bright nuclear staining (F).

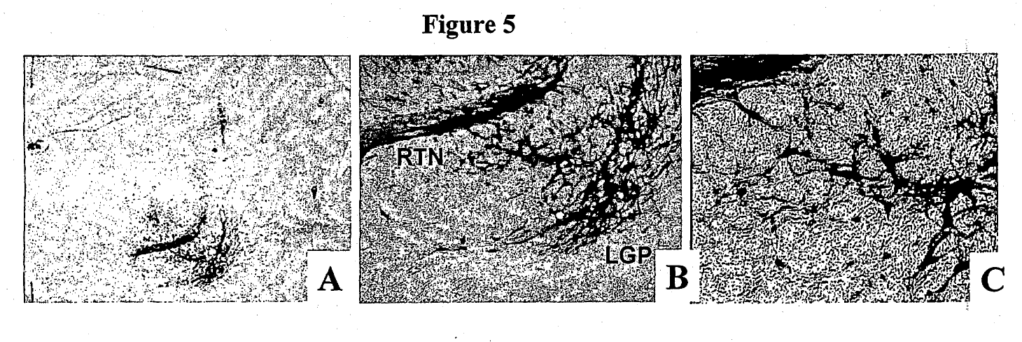

FIG. 5 shows the transduction of globus pallidus and reticular thalamic nucleus: (A) In rats where transduction with EIAV pONY8Z VSVG spread to lateral globus pallidus (LGP) LacZ staining is also observed in thalamic reticular nucleus (RTN). Higher magnification indicates the presence of efferent connections from GP passing along the zona incerta to RTN and thalamus (B,C). This anterograde transport is reported in other studies using specific anterograde tracers (Shammah-Lagnado et al J Comp Neurol 1996 376: 489-507).

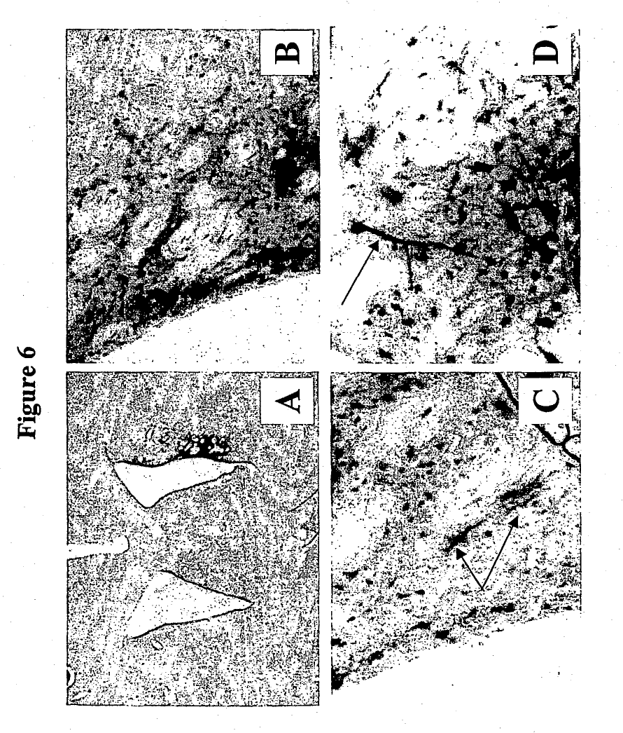

FIG. 6 shows the transduction of the adult rat striatum with EIAV pONY8Z RabiesG viral vector (A) Low magnification of brain section showing transduction in caudate adjacent to lateral ventricle. Higher magnifications of the same section show the punctate nature of expression (B) and transduction of cells with astroglial morphology (C arrows) as well as neuronal morphology (D arrow).

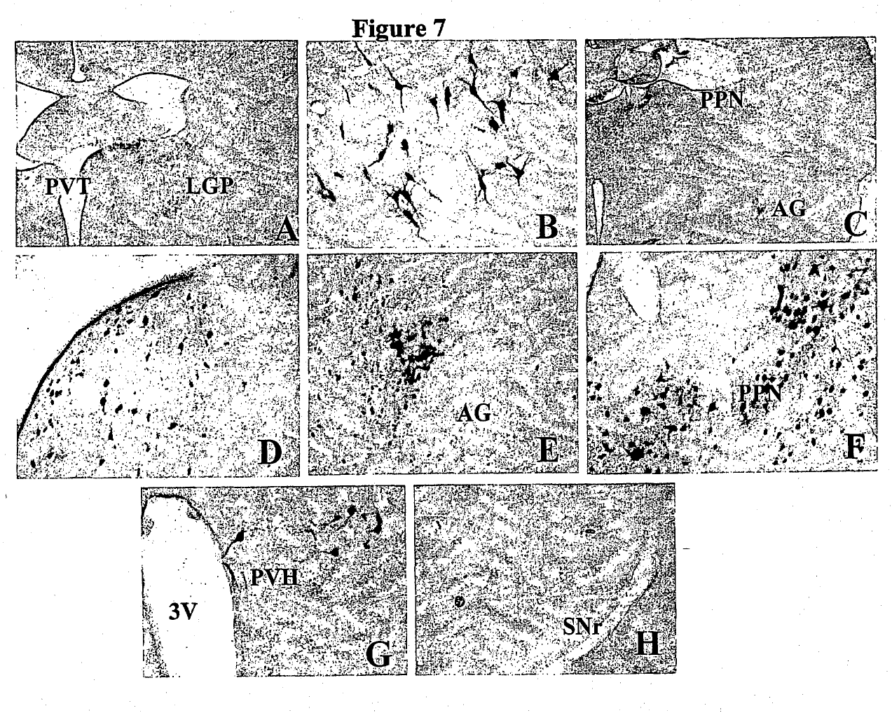

FIG. 7 shows the transduction of neuronal nuclei distant to the area of injection after delivery of EIAV pONY8Z RabiesG viral vector in adult rat striatum (8 days postinjection): (A) Low magnification image of brain section showing transduction in globus pallidus (LGP) and paraventricular nuclei of thalamus (PVT). (B) Higher magnification image of transduced pallidal neurons. (C) Low magnification image of brain section showing staining in paraventricular paracentral nucleus of stria terminalis and also staining in amygdala (ventral). (D) Higher magnification image of (A) punctate staining of paraventricular nucleus of thalamus (E) Higher magnification of (C) showing staining of neurons in amygdala (F) Stria terminalis staining in paraventricular nucleus thalamus (G) Hypothalamic neurons of the paraventricular nucleus staining adjacent to the third ventricle. (H) Neuronal staining in SN reticulata. Thalamic staining implies retrograde transport of viral particles from neuronal terminals to neuronal cell bodies.

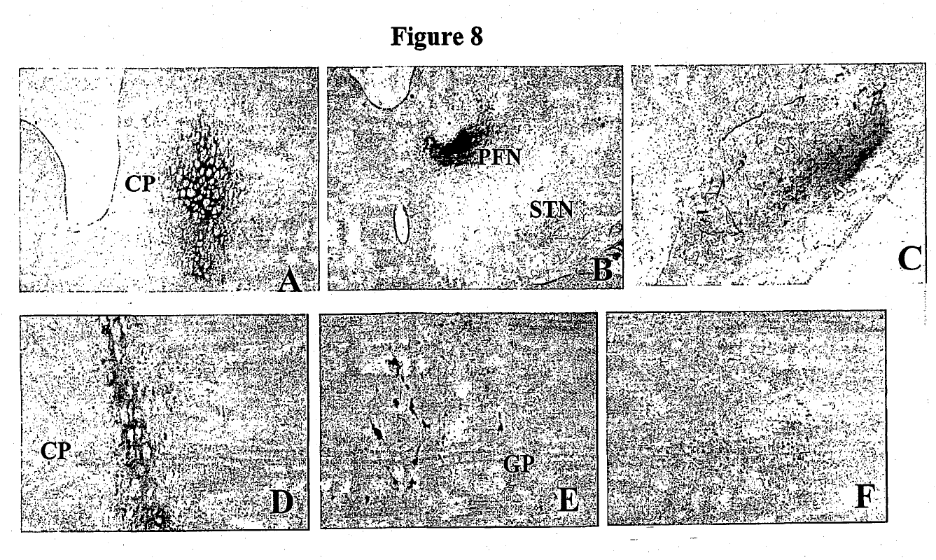

FIG. 8 shows long term expression of LacZ after transduction of the adult rat striatum with EIAV pONY8Z RabiesG viral vector (A,D) Striatal staining (B) Staining in parafascicular nucleus of thalamus (PFN) and weaker staining in subthalamic nucleus, (C) staining in SN compacta and reticulata, (E) neuronal staining in globus pallidus and (F) punctate staining of medial thalamic nuclei. (A,B,C) is expression after 3 months while (D, E, F) 6 months postinjection. Thalamic and SNc staining implies retrograde transport of viral particles from neuronal terminals to neuronal cell bodies.

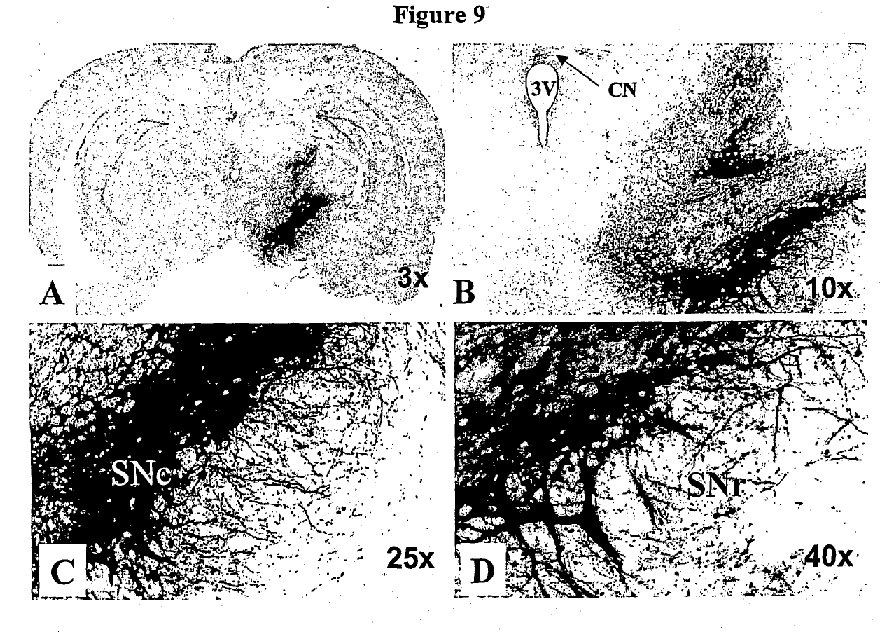

FIG. 9 shows the transduction of the adult rat substantia nigra with EIAV pONY8Z VSVG viral vector (A) Low magnification image showing spread of transduction after perinigral injection both in SNc, medial thalamus and hypothalamus (B) Higher magnification image showing neuronal transduction of thalamus with commissural neurons (CN) whose labelled axons cross dorsal to the third ventricle (3V) and terminate in contralateral thalamus. LacZ is transported in an anterograde manner in this case. (C,D) Higher magnification images of transduction of SNc showing stained neural projections from SNc to SNr. Transduction was 4 weeks postinjection.

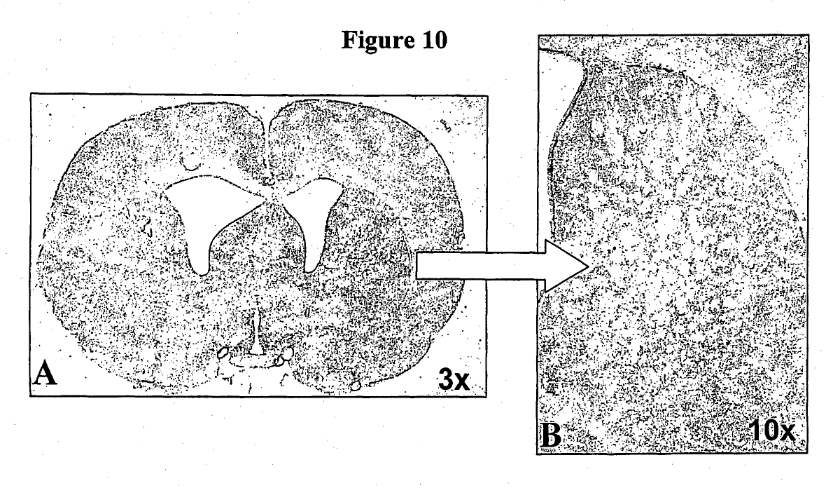

FIG. 10 shows anterograde staining of nigrostriatal terminals after perinigral injection of EIAV pONY8Z VSVG: (A) Low magnification image of brain striatal section from brain depicted in FIG. 9, showing LacZ staining of nigrostriatal terminals at the ipsilateral side of transduction. (B) Higher magnification image of anterograde transport of LacZ resulting in pale staining of neuronal terminals in striatum.

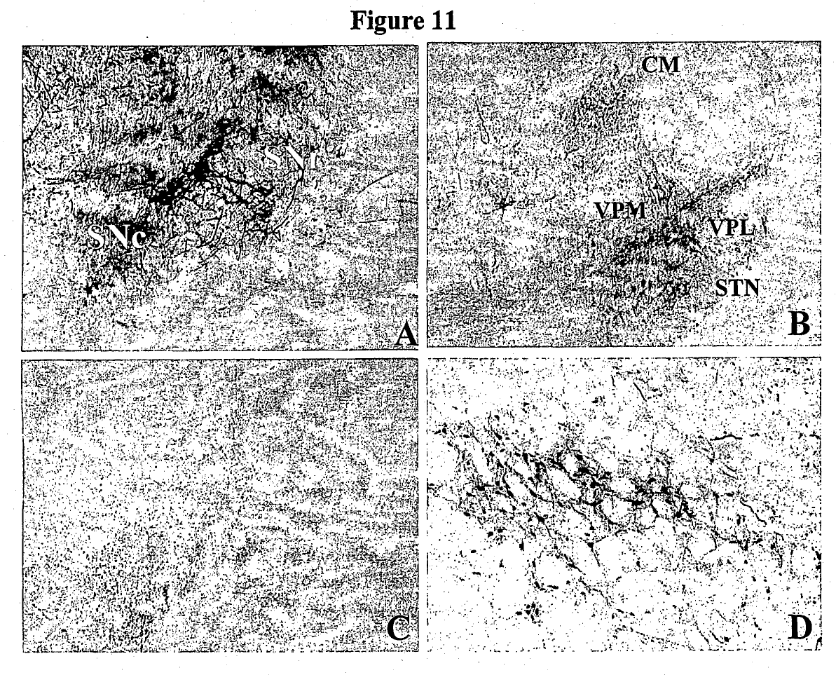

FIG. 11 shows transduction of the adult rat substantia nigra with EIAV pONY8Z Rabies G viral vector (A) Strong staining of neurons within SNc but also SNr. Also extensive spreading is observed in thalamus dorsal to SN. (B) Transduction of ventral posterolateral (VPL) and ventral posteromedial thalamic nuclei (VPM) (which receive input from medial lemniscus) centromedian nucleus (CM) and its thalamostriate fibers (wich project to putamen) and STN (which projects to medial GP and receives input from LGP) was observed on the ipsilateral side injection. (C) Punctate staining of putamen and cortex (pale staining indicative of neuronal terminals staining with LacZ transported anterogradelly) (D) Extensive transduction of neurons of globus pallidus (anterograde and retrograde transport). Transduction was 4 weeks postinjection.

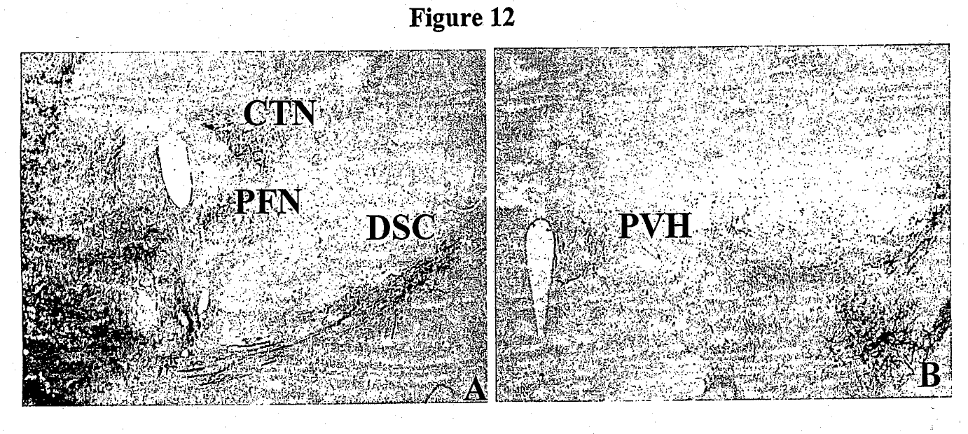

FIG. 12 shows staining after perinigral injection of EIAV pONY8Z Rabies G viral vector. (A) Staining of cell bodies of cental lateral (CLT) and parafascicular (PTN) thalamic nuclei as well of the dorsal supraoptic decussation of the commissure of Maynert (DSC) are staining at the contalateral side from the injection. The commissure of Maynert projects from STN contalateral to the side of injection to globus pallidus on the ipsilateral side. Since GP is transduced this staining implies retrograde transport of the vector to the neuronal bodies of the contalateral side. (B) Staining of paraventricular nucleus of hypothalamus (PVH) is seen as observed with VSVG pseudotyped vector (FIG. 7).

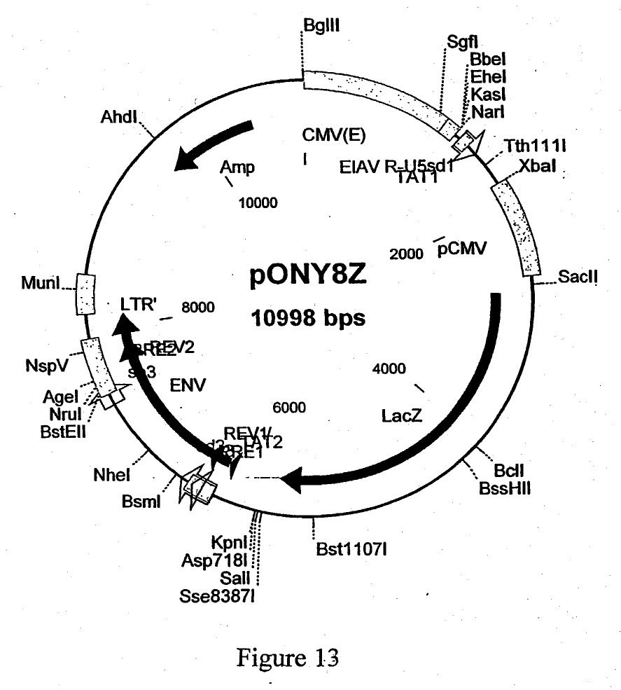

FIG. 13 shows a plasmid map of pONY8Z

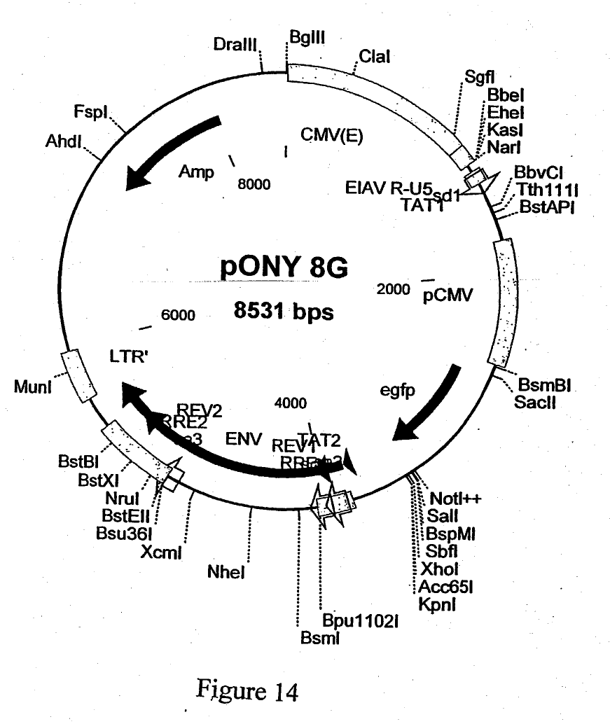

FIG. 14 shows a plasmid map of pONY8.0G

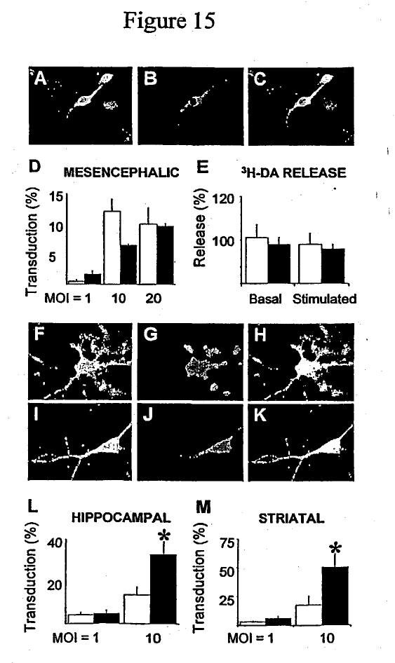

FIG. 15 shows gene transfer in primary neuronal cultures using EIAV lentiviral vectors. (A-C) Mouse E14 mesencephalic neurons infected with rabies-G pseudotyped pONY8.0G at an MOI of 10. A GFP expressing neuron from these cultures is shown in (A) labelled with an anti-GFP antibody and in (B) with an anti-tyrosine hydroxylase (TH) antibody. (C) GFP and TH colocalisation in the merged confocal image. (D) Increasing the MOI leads to an increase in the number of neurons transduced but no significant differences between the two pseudotypes is observed. (E) No effect of transduction on ³H-DA release by mesencephalic neurons after lentiviral gene transfer is observed compared to control neurons. In D, E, L & M clear bars indicate cells infected with VSV-G pseudotyped vector and black bars, cells infected with rabies-G pseudotyped vector. (F-H) Rat E17 hippocampal neurons and striatal neurons (I-K) infected with rabies pseudotyped EIAV vectors expressing βgal at an MOI of 10. Cells are labelled with anti-β-gal (F, I) and anti-Neuronal Nuclei (NeuN) antibodies. (G, J) Merged confocal images showing colocalization of the two antigens (H-K). As with the mesencephalic cultures, increasing MOI leads to an increase in the number of hippocampal (L) and striatal (M) neurons transduced. * indicates a significant increase in transduction efficiency with the rabies-G pseudotyped vector compared to the VSV-G pseudotype. Images A-C, & F-K: ×60 magnification.

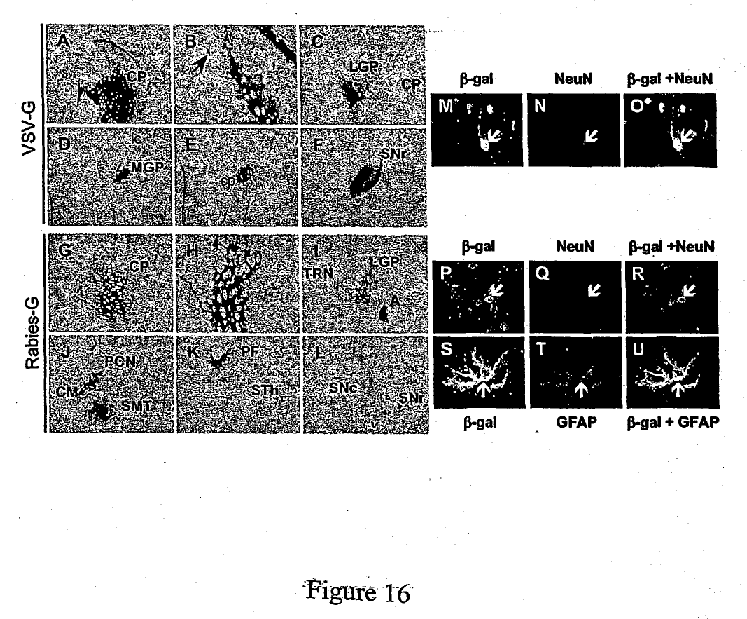

FIG. 16 shows in vivo transduction of LacZ in the rat striatum with VSV-G (A-F) and rabies-G (G-L) pseudotyped pONY8Z vectors 1 month post-injection. (A) Extensive gene transfer at the site of injection in the caudate putamen is observed after VSV-G pseudotyped vector delivery, which is specific to the striatum and not to the fiber tracts transversing it. (B) Higher magnification image from (A), revealing cells with neuronal morphology close to the injection site (arrow). Anterograde transport of β-gal is observed in neuronal axons projecting from the injected striatum to anatomically linked projection sites such as the lateral and medial globus pallidus (C, D), the cerebral penduncle adjacent to the subthalamic nucleus (E) and to the substantia nigra pars reticulata (F). The striatal projections to these sites are reviewed in (Parent et al (2000) Trends Neurosci 23 S20-7). Some β-gal expressing cell bodies are observed only in the lateral globus pallidus, which implies that direct gene transfer has also occurred due to the proximity of this nucleus to the injection site.

(G) Gene transfer with rabies-G pseudotyped vectors in striatum leads to extensive β-gal staining in caudate putamen (G, H) and also of the nearby globus pallidus (I). Pallidal transduction leads to anterograde labelling of projections to thalamic reticular nucleus (I). Labelling of these afferents was observed when anterograde tracers were placed in the globus pallidus. Retrograde transport of rabies-G pseudotyped viral vectors results in transduction of cell bodies in distal neuronal nuclei at anatomically connected sites including the amygdala. (I), several thalamic nuclei (J,K), the subthalamic nucleus (K) and the substantia nigra (L). This phenomenon was not observed after similar delivery of VSV-G pseudotyped vectors. Confocal analysis of transduced cell-types in the rat striatum following injection of VSV-G (M-O) and rabies-G (P-U) pseudotyped EIAV viral vectors. Transduction is mainly neuronal in both cases as demonstrated with β-gal (M and P) and NeuN antibody staining (N and Q) in the same sections. Colocalization of β-gal and NeuN expression can be seen in the merged images (O and R). Note transduced striatal projection neuron in the case of VSV-G (arrow) but absent in the striatum transduced with the rabies-G pseudotyped vector. In addition to neurons (arrow) rabies-G pseudotyped vector transduces astrocytes (S-U arrow), as demonstrated by anti-β-gal (S) and anti-GFAP (T) colocalisation (U). A: amygdala, CP: caudate putamen, cp: cerebral penduncle, CM: centromedial thalamic nucleus, ic: internal capsule, LGP: lateral globus pallidus, MGP: medial globus pallidus, PCN: pericentral thalamic nucleus, PF: perifasicular thalamic nucleus, SNc: substantia nigra pars compacta, SNr substantia nigra pars reticulata, SMT: submedial thalamic nucleus, STh: subthalamic nucleus, TRN: thalamic reticular nucleus. Images A,C, D,E,F,G,I,J,K: ×10; H: ×25; B: ×40; M-O: ×90; P-R: ×120; S-U: ×160 magnification.

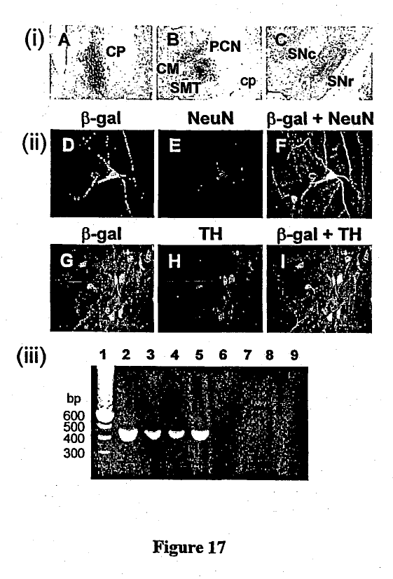

FIG. 17(I) shows reporter gene expression at eight months post-injection in the striatum and retrogradely transduced distal sites after striatal delivery of rabies-G pseudotyped pONY8Z vector. (A) Strong expression at the site of delivery in the caudate putamen. Expression also remains strong at distal sites projecting to caudate putamen, such as the medial thalalamic nuclei (B) and the substantia nigra (C), which are transduced by retrograde transport of the rabies-G pseudotyped pONY8Z vector. Pale staining is observed in cerebral penduncle and substantia nigra pars reticulata from β-gal transported in axons of transduced striatal efferents. CM: centromedial thalamic nucleus, CP: caudate putamen, cp: cerebral penduncle, PCN: pericentral thalamic nucleus, SMT: submedial thalamic nucleus, SNc: substantia nigra pars compacta, SNr substantia nigra pars reticulata. Images A, B: ×10; C: ×15 magnification.

FIG. 17 (II) shows confocal analysis showing retrogradely transduced neurons in globus pallidus (D-F) and substantia nigra pars compacta (G-I), after injection of rabies-G pseudotyped vector into the striatum. Micrographs demonstrate immunofluorescent labelling of neurons with anti-β-gal (D and G), anti-NeuN (E) and anti-tyrosine hydroxylase (H) antibodies. Expression of β-gal colocalizes with the immunofluorescence of NeuN in pallidal neurons (F) and tyrosine hydroxylase in nigral dopaminergic neurons (I), producing bright staining. Images D-I: ×50 magnification.

FIG. 17 (II) shows PCR analysis showing detection of EIAV vector DNA in thalamus and substantia nigra ipsilateral to the site of injection of the rabies-G pseudotyped vector in the rat striatum. Lane 1: 100 bp ladder; Lanes 2, 3, 4: Rat 1 (rabies-G pseudotyped vector) striatum, thalamus, substantia nigra; Lanes 5, 6, 7: Rat 2 (VSV-G pseudotyped vector) striatum, thalamus, substantia nigra; Lane 8: Rat 5 uninjected; Lane 9: water.

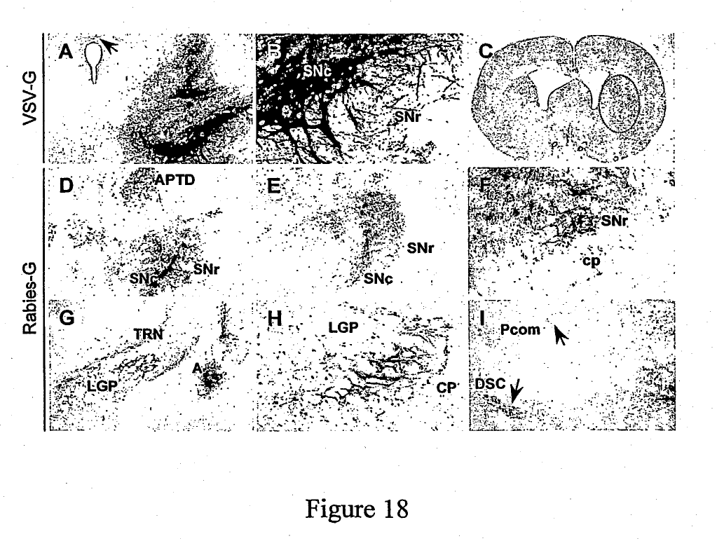

FIG. 18 shows in vivo transduction of LacZ in the rat substantia nigra with VSV-G (AC) and rabies-G (D-I) pseudotyped pONY8Z vectors 1 month post-injection. (A) Extensive gene transfer is observed with the VSV-G pseudotyped vector in the substantia nigra pars compacta and thalamus. (B) Higher magnification of the substantia nigra showing extensive transduction of pars compacta neurons and their axons projecting to substantia nigra pars reticulata. (C) β-gal protein is anterogradely transported to axon terminals of nigrostriatal neurons producing pale staining of ipsilateral striatum (encircled). Arrow in (A) indicates anterograde transport of β-gal and staining of commisural axons projecting to contralateral side though no transduction of neuronal cell bodies was observed-contralaterally. (D) Extensive transduction of both substantia nigra and different thalamic nuclei is observed after delivery of rabies-G pseudotyped EIAV vectors. In this case both substantia nigra pars compacta and substantia nigra pars reticulata are transduced (E,F). Labelling of neurons in distal sites due to retrograde transport of this vector can be observed in lateral globus pallidus (G,H), amygdala (G) and commissural neurons projecting from contralateral thalamus (arrows 1). Anterograde transport of β-gal along axons is widespread, leading to staining of structures such as the thalamic reticular nucleus (G) (from lateral globus pallidus) and caudate putamen (G,H) (from substantia nigra pars compacta and lateral globus pallidus). A: amygdala, APTD: anterior pretectal thalamic nucleus, CP: caudate putamen, cp: cerebral pendunde, DSC: dorsal supraoptic decussation of the commissure of Maynert, LGP: lateral globus pallidus, PCom: nucleus of posterior commissure, SNc: substantia nigra pars compacta, SNr: substantia nigra pars reticulata, TRN: thalamic reticular nucleus. Images C: ×3.5; A,D,E,G,I: ×10; F,H: ×25; B: ×40 magnification.

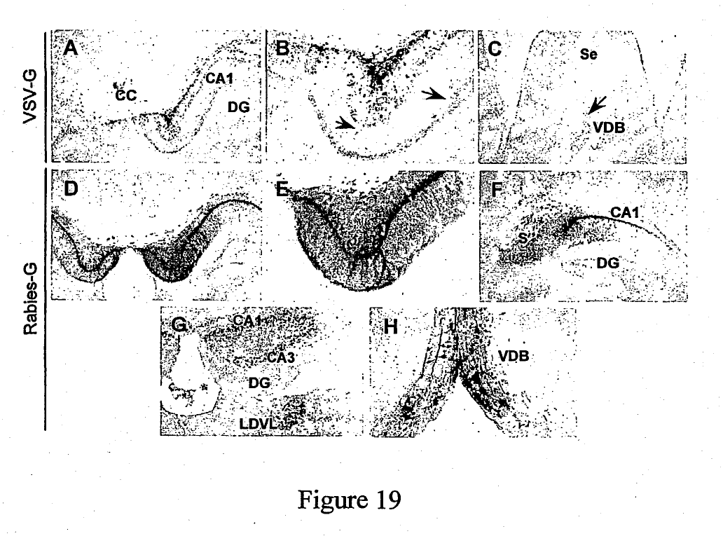

FIG. 19 shows in vivo transduction of LacZ in the rat hippocampus with VSV-G (AC) and rabies-G (D-H) pseudotyped pONY8Z vectors 1 month post-injection. (A) Extensive gene transfer is observed with the VSV-G pseudotyped vector in the subiculum and to a lesser extent in the CA1 pyramidal cell layer and in the corpus callosum. Faint blue staining represents anterograde transport of β-gal staining of axon fibers projecting to the stratum moleculare (A & B arrows) and a few fibers projecting to the septum and diagonal band of Broca (C arrow). No cell body staining was observed in these regions. These neuronal projections are established from anterograde tracing experiments. (D) Strong transduction of CA1 cells with rabies-G compared to VSV-G pseudotyped vectors is observed. Some transduction of CA4 pyramidal cells is also present (E) Higher magnification from the CA1 region depicted in (D) showing strong staining of apical dendrites and axons of pyramidal neurons. (F) B-gal staining of cells in the subiculum, CA1 pyramidal layer, corpus callosum and cortical fibers in the posterior hippocampus. (G) β-gal staining of CA1 and CA3 pyramidal cells but not of dentate gyrus in the anterior hippocampus. Cortical fibers are stained and retrograde labelling of laterodorsal thalamic nucleus is also observed. (H) Strong transduction in neuronal nuclei and axons in the lateral hypothalamus and diagonal band of Broca, due to retrograde transport of the rabies-G pseudotyped viral vector is observed. Afferents to the hippocampus from these sites have been previously described. DG: dentate gyrus; CA1,CA3: hippocampal pyramidal neuronal cell layers; LDVL; vetrolateral aspect-of-laterodorsal-thalamic nucleus; S: subiculum; Se: septum; VDB: vertical limb of the diagonal band of Broca.

Images A,C,D,F: ×10; G: ×15; B,H: ×25; E: ×50 magnification.

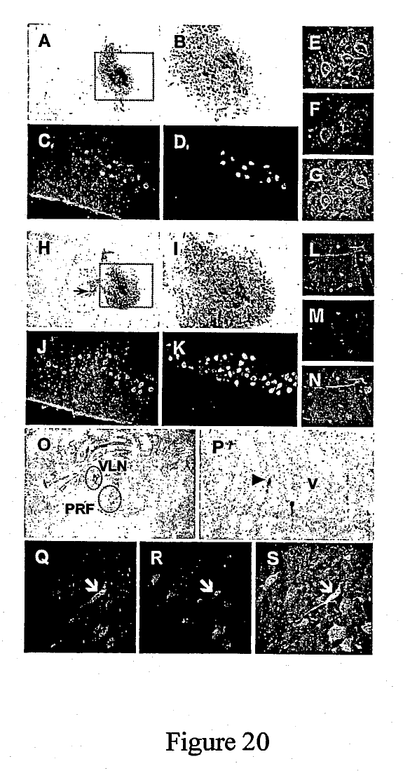

FIG. 20 shows reporter gene expression in the rat spinal cord 3 weeks following intraspinal or intramuscular delivery of pONY8Z lentiviral vectors. Micrographs of the ventral horn showing transduction after intraspinal injections with VSV-G (A-G) or rabies-G pseudotyped vector (H-P). Strong transduction with β-gal is observed with both types of vectors (A-B; H-I). B and I are higher magnifications from the area of transduction shown in A and H. Longitudinal sections of the spinal cord showing retrogradely fluorogold-labeled motoneurons (D & K) co-expressing β-gal (C & J).

Transverse sections stained with anti-β-al antibodies (E, L, Q). Same sections stained for the neuronal marker NeuN (F, M, R). Composite confocal images showing neuronal colocalisation of NeuN and β-gal (G, N, S). Retrogradely transduced motoneurons are observed in areas projecting to the site of injection such as brainstem (O) and layer V of the cerebral cortex (P) following intraspinal injection of rabies-G pseudotyped pONY8Z vectors. Arrow in H indicates retrogradely transduced commissural motoneurons projecting from the contralateral side to the region of injection, along previously established anatomical connections. The arrowhead in P indicates a transduced layer V corticospinal motoneuron ipsilateral to the injection site. (Q-S) Transverse sections of the spinal cord showing retrograde transport of the viral particles and transduction of spinal cord motoneurons (arrow) after injection of rabies-G pseudotyped pONY8Z vector from the gastrocnemius muscle. Sections stained with anti-β-gal antibodies (Q). Same sections stained for the neuronal marker NeuN (R). Composite confocal images showing colocalisation of NeuN and β-gal (S). Vln: vestibular lateral nucleus; Prf: pontine reticular formation. Images A,H: ×10; B-D, I-K, & O: ×25; P: ×50; E-G, L-N & Q-S: ×60 magnification.

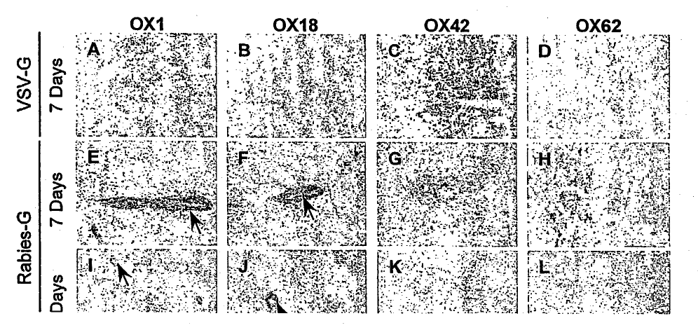

FIG. 21 shows the immune response in the rat brain following pONY8Z vector delivery in the rat striatum. Antibodies used to detect components of the immune response in the injected area were as follows: OX18—leucocyte common antigen, OX18—MHC class 1, OX42—complement receptor type 3 on microglia and macrophages and OX62—dendritic cells. All animals (including PBS-injected controls, not shown) exhibited a minor infiltration of OX42⁺/ED1⁺ activated macrophages/microglia around the needle tract in the cortex and striatum (C, G, K). This response declined with time but was still partially evident at 35 days postinjection (not shown). Animals injected with VSV-G pseudotyped vectors (A-D) exhibited a minor immune response at 7 days post-injection, in addition to the microglial infiltration observed in controls. An infiltration of OX18+ MHC class I positive cells in ipsilateral striatum (B) was observed though neither leucocytes (A) nor dentritic cells (D) could be detected at any time after VSV-G pseudotyped vector injection in the brains of these animals. This response had declined by 14 days. Compared to VSVG pseudotyped vector, a slightly stronger immune response was observed following injection of rabies-G pseudotyped vector. Infiltration of leucocytes (E), MHC class I immunopositive cells (F) dendritic cells (H) and the presence of perivascular cuffing (E, F) can be seen 7 days after injection, decreasing in levels at 14 days after injection (I, J, K respectively). Images A-D & F-L: ×25; E: ×50 magnification.

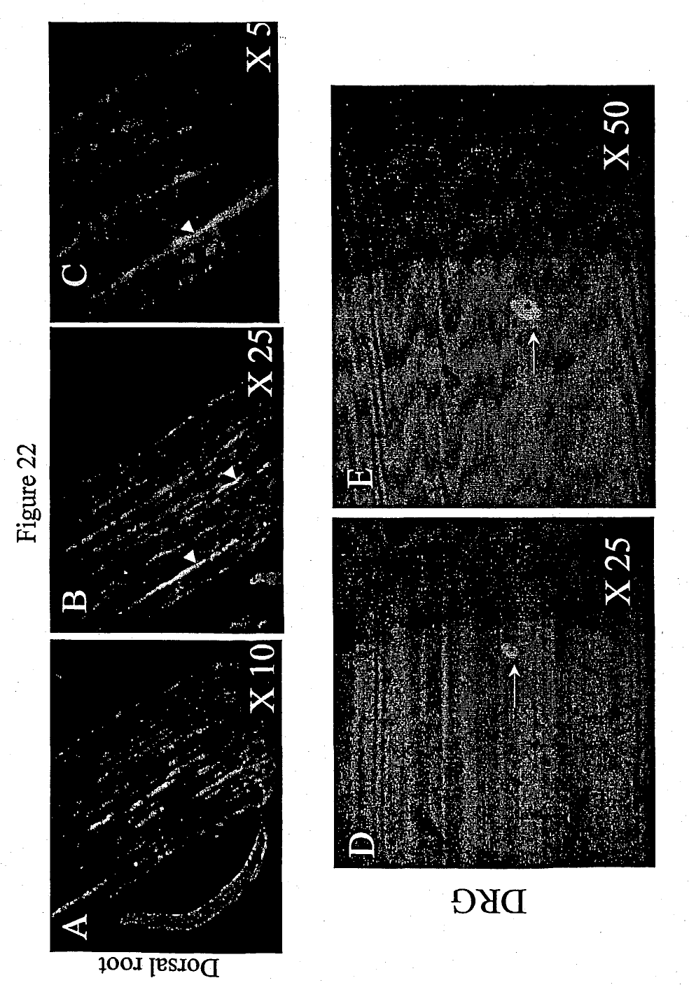

FIG. 22 shows viral transfer of genes to sensory neurons. Expression of the reporter gene β-galactosidase in the dorsal root (A-C) and DRG (D, E) after injection of pONY8Z pseudotyped with rabies-G into the dorsal horn of the spinal cord. Sections showing immunofluorescence for β-galactosidase 5 weeks after viral injections. Expression of β-gal is detectable in Shwann cells, axons (block arrow) and DRG neurons (arrow). For immunofluorescence, sections were incubated with rabbit polyclonal anti-β-gal (5Prime3Prime Inc.) at dilution of 1:250. The second antibody used in this expreriment was FITC-conjugated anti-rabbit IgG (Jackson lmmunoresearch).

### EXAMPLES

### Example 1

### Transduction of Presumptive Dopaminergic (TH+) Neurons in Rodent Mesencephalic Cultures

Methods Mesencephalic cultures: Cultures are prepared exactly as described by Lotharius et al. (1999) (J. NeuroSci. 19:1284-1293). Briefly, the ventral mesencephalon was removed from embryonic day 14 (E14) CF1, murine embryos (Charles River Laboratories, Willington, Mass.). Tissues are mechanically dissociated, incubated with 0.25% trypsin and 0.05% DNase in phosphate buffered saline (PBS) for 30 minutes at 37° C., and further triturated using a constricted Pasteur pipette. For immunocytochemistry, cells are plated at a density of 50,000 cells per 35 mm microwell plate (1.25×103 cells/mm²). All plates are pre-coated overnight with 0.5 mg/ml poly-d-lysine followed by 2.5 mg/ml laminin for 2 hours at room temperature. Initial plating is done in serum-containing medium consisting of 10% fetal calf serum in DMEM:F1 supplemented with B27 additive (Life Technologies, Gaithersburg, Md.), 6 g/L glucose, and antibacterial agents. Glial numbers are reduced by subsequently maintaining, cells in serum-free Neurobasal medium (Life Technologies) supplemented with 0.5 mM L-glutamine, 0.01 mg/ml streptomycin/100 units penicillin, and 1X B27 supplement. Half of the culture medium is replaced with fresh Neurobasal medium every 48 hours.

DA Release: In order to measure dopamine uptake, release and content cells are plated at a density of 400,000 cells per 16 mm well (2×103 cells/mm²). To measure DA release, cells are loaded with 2.4*Ci/ml 3H-DA/KRS for 20 min at 37° C. and washed 3×3 min. Radioactive counts from a wash sample is measured using a Beckman scintillation counter and used as a control for basal levels of 3H-DA release. Cells are then treated with 30 mM K+ in KRS (adjusted as described in Dalman & O'Malley, 1999 J. Neurosci 19:5750-5757) for 5 min and the amount of 3H-DA released during this time period is collected. Subsequently, cultures are washed extensively and lysed in 0.1 N PCA by freeze-thawing, and residual, intracellular 3H-DA is measured. Total 3H-DA uptake is calculated by summation of tritium content from all of the fractions collected, including the acid lysate.

Plasmid construction:

a) Vector Plasmids

Numbering used is as of Payne et al 1994 (J. Gen Virol. 75:425-429). The pONY series of vectors and their pseudotyping with the different envelopes have been described previously (WO99/61639) (Mitrophanous et al. 1999 Gene Ther 1999 6:1808-1818). pONY8Z (FIG. 13, SEQ ID No 1) was derived from pONY4.0Z (WO99/32646) by introducing mutations which prevented expression of TAT by an 83nt deletion in the exon 2 of tat, prevented S2 expression by a 51nt deletion, prevented REV expression by deletion of a single base within exon 1 of rev and prevented expression of the N-terminal portion of gag by insertion of T in the first two ATG codons of gag, thereby changing the sequence to ATTG from ATG. With respect to the wild type EIAV sequence (Acc. No. U01866) these correspond to deletion of nt 5234-5316 inclusive, nt 5346-5396 inclusive and nt 5538. The insertion of T residues was after nt 526 and 543. pONY8.0G (FIG. 14, SEQ ID No 2) was derived from pONY8Z by exchange of the Lac Z reporter gene for the enhanced green fluorescent protein (GFP) gene. This was done by transferring the Sac II-Kpn I fragment corresponding to the GFP gene and flanking sequences from pONY4.0G (WO099/32646) into pONY8Z cut with the same enzymes.

b) Envelope Plasmids

pSA91ERAwt was used for pseudotyping with rabies G. This plasmid has been described previously (WO99/61639) under the name “pSA91RbG”. Briefly, pSA91ERAwt was constructed by cloning 1.7 kbp Bg/II rabies G fragment (strain ERA) from pSG5rabgp (Burger et al., 1991 J. Gen. Virol. 72. 359-367) into pSA91, a derivative of pGW1HG (Soneoka et al 1995 Nucl. Acids Res. 23: 628-633) from which the gpt gene has been removed by digestion with BamHI and re-ligation. This construct, pSA91 ERAwt, allows expression of rabies G from the human cytomegalovirus (HCMV) immediate early gene promoter-enhancer.

pRV67 was used for pseudotyping with rabies G. pRV67 (described in WO99/61639) is a VSV-G expression plasmid in which VSV-G was expressed under the control of human cytomegalovirus promoter/enhancer, in place of rabies G in pSA91ERAwt.

Production and Assay of Vectors: Vector stocks were generated by calcium-phosphate transfection of human kidney 293T cells plated on 10 cm dishes with 16 jig of vector plasmid, 16 μg of gag/pol plasmid and 8 μg of envelope plasmid. 36-48 h after transfection, supernatants were filtered (0.45 μm) aliquoted and stored at −70° C. Concentrated vector preparations were made by initial low speed centrifugation 6 000× g (JLA-10.500 for 16 hours at 4° C. followed by ultracentrifugation at 20 000 rpm (SW40Ti rotor) for 90 min, at 4° C. The virus was resuspended in PBS for 3-4 h aliquoted and stored at −70° C. Transduction was carried out in the presence of polybrene (8 μg/ml).

Viral transductions: Transductions are carried out after 7 days in vitro (DIV7). Specifically, culture media are removed and reserved with a small aliquot being added back to cultures following the addition of the indicated viral MOI. Dishes were maintained at 37° C. for 5 hours after which the virus is removed and the wells are washed twice with the reserved conditioned media. Fresh Neuralbasal media is added in a 50:50 ratio and cells are maintained for a further 3 days.

Immunocytochemistry: To determine the effect of viral transductions on dopaminergic cultures plates are processed for TH and GFP immunoreactivity. Briefly, cells were rinsed with PBS, fixed in 4% paraformaldehyde, permeabilized in 1% bovine serum albumin/0.1% Triton-X-100/PBS for 30 minutes at room temperature (RT), and incubated with a mouse monoclonal anti-TH antibody (1:1000; Diastor) as well as a rabbit polyclonal anti-GFP antibody (1:1000; Chemicon) for 1 hr at 37° C. Cells are subsequently incubated with a CY3-conjugated anti-mouse IgG (1:250; Jackson Immunoresearch) and an Alexa-488-conjugated anti-rabbit secondary (1:250; Molecular Probes). Neurons are imaged with a Fluoview confocal microscope (Olympus America Inc). Manual cell counts were conducted as described (Lotharius et al, 1999). Briefly, 6 consecutive fields are assayed per dish leading to the quantification of 200-300 TH neurons per experiment. Experiments are repeated 3 times using cultures isolated from independent dissections. Descriptive statistics (mean± SEM) of cell counts are calculated with statistical software (GraphPad Prism Software Inc.)

Results

a) Comparison of transduction with EIAV vectors Pseudotyped with VSVG and Rabies G

In order to determine whether the equine lentiviral preparations could transduce TH+ neurons in vitro, mesencephalic cultures were prepared and transduced on DIV7. This time point was chosen because it had been previously determined that most characteristic dopaminergic functions were established by then (Lotharius et al., 1999 as above; Dalman and O'Malley, 1999 as above; Lotharius and O'Malley, 2000 J. Biol. Chem. e-publication (ahead of print) 31 August 2000). Both pSA91ERAwt and pRV67 pseudotyped EIAV vectors were capable of transducing dopaminergic neurons in vitro at about 10% efficiency at the highest MOI tried (Table 1, FIG. 1 and FIGS. 15A-D). Both vectors also transduced non-dopaminergic neurons and glial populations as judged by morphological criteria (FIG. 2). In particular the pRV67 vector transduced approximately 80% of the estimated glia/per dish whereas the pSA91 ERAwt vector transduced only 5-10%.

TABLE 1
Transduction efficiency of dopaminergic neurons in vitro

| | pSA91ERAwt | pRV67 |
|---|---|---|
| MOI 1 | 1.7 ± 0.50* | 0.5 ± 0.30 |
| MOI 10 | 6.5 ± 0.16 | 12.1 ± 2.0 |
| MOI 20 | 9.7 ± 0.42 | 10.0 ± 2.7 |

*SEM

b) Functional Analysis of Transduced Cultures Using Uptake and Release of Dopamine Assay

To determine whether viral transduction altered dopaminergic properties the 3H-dopamine (³H-DA) release assay was used. Because dopamine transporters are localized exclusively on dopaminergic neurons in the midbrain (Kuhar et al., 1998 Adn. Pharmacol. 42:1042-5), this approach allows for the selective analysis of dopaminergic function in the midst of a heterogeneous culture system. The data indicate that neither pSA91ERAwt nor pRV67 pseudotyped vectors affected 3H-DA release (Table 2 and FIG. 15E) and this is indicative of not causing an aberration in the function of the TH+ neurons after EIAV vector transduction.

TABLE 2
Effects of viral transduction on DA uptake and release

| | pSA91ERAwt | pRV67 |
|---|---|---|
| Stage | % control | % control |
| Basal Release | 98 ± 3 | 101 ± 6 |
| K ± stimulated | 96 ± 2 | 98 ± 5 |

Cultures are kept naive or are transduced with the indicated viral particles at an MOI of 20 as described in the Methods. Following transduction the media is removed, and the cultures are washed with KRS and then loaded with 3H-DA. Basal or spontaneous release is measured at 10 min after exposure to 3H-DA. Release is expressed as a percentage of total uptake SEM. Typically basal release is 2-3% of the total and K ± stimulated release was 5-6% of the total uptake.

Primary cultures of both hippocampal and striatal neurons could also be transduced in vitro by EIAV vectors pseudotyped with either VSV-G or rabies-G. This was demonstrated in hippocampal and striatal neurons by the colocalization of antibody staining for both the reporter protein β-gal and NeuN, a neuronal-specific marker (FIGS. 15F-H and I-K, respectively). At MOIs of 1 and 10, there was no significant difference in transduction efficiency between the hippocampal and striatal neurons (MOI=1, P=0.23 and MOI=10, P=0.81, ANOVA, FIGS. 15L & M), although an increase was observed compared to mesencephalic dopaminergic neurons. Similarly, there was no significant difference in transduction efficiency at MOI=1 when vectors are pseudotyped with either VSV-G or rabies-G (P=0.14, ANOVA). However, at an MOI of 10, the transduction efficiency of the rabies-G pseudotyped vector was significantly higher than that observed with the VSV-G pseudotyped vector (P<0.001, ANOVA).

### Example 2

### Transduction of the Adult Rat CNS

Methods

Stereotactic injection into rat brain In order to examine virally encoded gene expression EIAVlacZ (pONY8Z) pseudotyped with either VSV-G (pRSV67) or Rabies G (pSA91 ERAwt) are stereotaxically microinjected into the adult rat striatum as follows: rats are anesthesized with hypnorm and hypnovel (Wood et al., (1994) Gene Therapy 1:283-291) and injected with 2×1 μl of viral stocks (for EIAV IacZ is typically 1-5×10⁹ t.u./ml for VSV-G and 6×10⁸ t.u./ml for Rabies-G pseudotyped vector) into the striatum, at coordinates: Bregma 3.5 mm lateral, 4.75 mm vertical from dura, and 1 mm rostral, 3.5 mm lateral 4.75 mm vertical using a fine drawn glass micropippette over a period of 2 min. For perinigral (medial lemniscus) injections 2×1 μl of viral stocks were delivered at coordinates: 4.7 mm caudal to Bregma, 2.2 mm lateral, 7 mm vertical from dura and 5.4 caudal, 2.2 lateral and 7.5 mm vertical. The pippette was pulled up 1 mm and left for another 2 min before retracting slowly to the surface. Animals are analysed 1 and 2 weeks following injection. Rats are perfused with 4% paraformaldehyde (PFA) containing 2 mM MgCl₂ and 5 mM ethylene glycol bis (beta-aminoethylether)-N,N,N′,N′-tetraacetic add. At different time intervals after the intracranial injections rats are sacrificed brains are removed and placed in fixative overnight, submersed in 30% sucrose at 4° C. overnight and frozen on Tissue-Tech OCT embedding compound (Miles IN USA). Fifty-micrometer sections are cut on a freezing microtome and floated briefly in PBS-2 mM MgCl₂ at 4° C. as a wash. Expression of lacZ is determined by placing the sections in X-gal staining solution for 3-5 hours.

Immunohistochemistry: To determine whether the cells transduced are neurons or glial-cells a LacZ antibody is used in conjuction with antibodies that recognise either neuronal (NeuN) or glial (GFAP) markers. Double immunostaining is carried out on brain sections. Sections are incubated with rabbit polyclonal LacZ antibody (1/100^{th}&; 5 prime→3 prime) and mouse monoclonal neurofilament (NeuN) antibody (1/50^{th}; Chemicon), or mouse monoclonal GFAP (1/50^{th}; Chemicon) at 4° C. overnight in PBS-10% goat serum and 0.5% TritonX-100. Sections are washed with PBS and then incubated with Alexa 488 conjugated goat anti rabbit IgG (1/200^{th}; Molecular Probes) or Texas Red-X conjugated goat anti-mouse IgG (1/200^{th}; Molecular Probes) at room temperature for 2-3 hours. After washing the sections are examined under a fluorescence microscope.

Polymerase Chain Reaction

To detect viral DNA after injection of pONY8Z virus pseudotyped with VSV-G or rabies-G into rat striatum (n=4) (as described above), animals are sacrificed 2 weeks post-transduction. Punches from striatum, thalamus and substantia nigra are quickly removed and frozen in liquid nitrogen. Genomic DNA is isolated from all samples using the Wizard Genomic DNA Purification kit (Promega, Madison-Wisconsin # A1120). Thawed brain tissue (20 mg) is homogenized for 10 seconds using a disposable homogenizer in cooled nuclei lysis solution according to the manufacturer's protocol. PCR reactions are set to detect the E. coli LacZ gene (Gene Bank # V00296) expressed by injected vectors. Each reaction is set in 50 μl volume containing the following components (final concentration): 300 nM forward primer CGT TGC TGC ATA AAC CGA CTA CAC (nt: 638-661), 300 nM reverse primer TGC AGA GGA TGA TGC TCG TGA C (nt 1088-1067) 200 μM of dNTP (each), 2 mM MgCl₂, 1× FastStart Taq DNA polymerase buffer and 2 Units FastStart Taq DNA polyerase (Roche Diagnostics, Mannheim Germany). 300 ng of template DNA is used per reaction. PCR amplification is carried out on a PCR Express (Hybaid, Hercules, USA) under the following thermal cycling conditions: initial denaturation and enzyme activation at 95° C. for 4 minutes followed by 30 cycles of denaturation at 95° C. for 30 seconds, annealing at 58° C. for 45 seconds and elongation at 72° C. for 45 seconds and finally one cycle of extension at 72° C. for 7 minutes. PCR products (10 μl/reaction) are resolved on 1.2% TBE agarose gel at 10 v/cm for 2 hours.

Results

a) Comparison of Transduction Using EIAV Vectors Pseudotyped With VSVG and Rabies G after Delivery to Striatum.

In order to compare the pattern of expression bf the two different pseudotyped vectors in the adult rat brain, concentrated viral vector preparations are sterotactically injected into caudate putamen. VSVG pseudotyped EIAV-LacZ expressing vectors gave very efficient gene transfer spanning an average region of 2.5 mm anteroposterior (50×50 μm coronal sections stained), 1 mm mediolateral and 5 mm dorsoventral around the area of injection, giving an approximate cell volume transduced of ˜5×10⁴ (FIG. 3). This equates to about 29750±1488 transduced cells (FIGS. 16A & B). The transduced cells have principally neuronal morphology (striatal interneurons, medial spiny neurons and aspiny neurons) and this is further confirmed using confocal co-localisation of the neuronal marker NeuN and LacZ markers (FIG. 4 and FIGS. 16M-O). Transduced glia are seen in some rats in white matter tracts such as corpus callosum. Transduction is localised to striatum with some anterograde transport of LacZ proteins to axons projecting to subthalamic nucleus (SN), the lateral and medial globus pallidus (FIGS. 16C-D), cerebral penduncle (FIG. 16E), and the substantia nigra pars reticulata (SNr) (FIG. 16F). In rats where lateral globus pallidus (GP) is co-transduced reticular thalamic nucleus (RTN) is also strongly stained by anterograde transport of LacZ (FIG. 5).

Transduction of rat striatum with Rabies G pseudotyped EIAV-LacZ expressing vectors also gave efficient gene transfer to cells of both neuronal and glial phenotype within caudate putamen (FIGS. 16G-H). In addition a far greater spread of transduced neurons is observed in regions caudal to the site of injection including globus-pallidus, thalamus, amygdala, ventral tegmental area (VTA), subthalamic nucleus (STN) and substantia nigra compacta (SNc) and reticulata (SNr) (FIGS. 6-8, FIGS. 16G-L). Anatomical connections are known to exist between these structures (see, for example, “Human Anatomy” 1976 Carpenter M. B. Williams and Wilkins Co. Baltimore, 7^{th} Edition, and references therein). Average transduction is seen anteroposteriously (7.5 mm anteposterior to the injection site) in 60×50 μm coronal sections spanning striatum and also in neurons in 55×50 μm sections spanning GP and thalamus and also in 40×50 μm sections spanning SN. This is the result of retrograde transport of viral vector to neurons in these areas from their axon terminals in striatum as well as anterograde transport of LacZ to neuron terminals whose cell bodies are in striatum. Cell counts indicate that 32650±1630 cells were transduced in striatum, while 14880±744 neurons in thalamus and 3050±150 neurons in substantia nigra. Staining in caudate putamen is paler and more punctate in comparison to VSVG vectors, with approximately 80% neurons and 20% glia transduced (FIGS. 16P-U). Only glial cells appear to be completely stained with LacZ. In comparison neurons in other areas such as GP, VTA and SNr do stain in their entirety with LacZ (FIGS. 7-8).

Confocal colocalization studies at the injection site indicate that the glia transduced were astrocytes. No projection neurons were transduced in contrast with the VSV-G pseudotyped vectors. Anterograde transport of β-gal was also present in neurons transduced with the rabies-G pseudotyped vectors as indicated by the pale staining of the thalamic reticular nucleus (from lateral globus pallidal neurons) and the substantia nigra pars reticulata (from striatal neurons) (FIGS. 16I & L). Confocal studies confirmed the neuronal nature of the cells transduced distally when rabies-G pseudotyped vectors were delivered in to the caudate putamen, such as the NeuN positive pallidal neurons and the tyrosine hydroxylase positive dopaminergic neurons of the substantia nigra (FIGS. 17ii D-I).

Retrograde transport of viral vector itself was confirmed by PCR experiments using punches taken from thalamus and substantia nigra areas since viral DNA in these areas could only be detected after rabies-G pseudotyped EIAV striatal transduction (FIG. 17iii). Control experiments where integrase mutant viral preparations or vector preparations preheated at 50° C. were injected in the brain, failed to give any significant levels of transduction thus excluding the possibility that pseudotransduction was responsible for the observed gene transfer (Hass et al (2000) Mol Ther 2,71-80).

Long-term expression was observed after delivery of both types of vectors to the caudate putamen from 1 week for up to eight months post-injection in the present study (not all data shown). Expression of rabies-G pseudotyped vectors was observed both at the site of injection and at all the distal neurons that were transduced at one month post-injection (FIG. 17i A-C only thalamus and substantia nigra are shown).

b) Comparison of Transduction Using EIAV Vectors Pseudotyped with VSVG and Rabies G to substantia nigra

In order to compare the ability of the two different pseudotyped vectors to transduce central nervous system dopaminergic neurons, concentrated viral vector preparations are stereotactically injected in the vicinity of substantia nigra (medial lemniscus). Perinigral injections are preferable since SN is prone to cell death after damage. VSVG pseudotyped EIAV-LacZ expressing vectors gave very efficient transduction of SNc and the thalamic structures caudal to it (FIG. 9 and FIGS. 18A and B). LacZ is transported anterogradely to axon terminals of the nigrostriatal neurons giving staining in striatum (FIG. 10 and FIG. 18C). Projections of neurons from SNc to SNr are also stained. LacZ staining spanned 40×50 μm coronal thalamic/nigral sections.

In contrast perinigral injections of Rabies-G pseudotyped EIAV vector gave strong transduction of SNc neurons and much wider transduction of rostal thalamic nuclei and in addition transduction was observed in neurons of the SNr, STN, VrA, thalamus, GP and cortex (FIGS. 11, 12). The β-gal staining was observed with the VSV-G pseudotyped vectors and in addition many fibres within the thalamus were stained. Transduction of distal neurons in the lateral globus pallidus and amygdala, where stronger β-gal staining was observed, was due to retrograde transport of virus from efferent connections to the substantia nigra pars reticulata and pars lateralis, respectively (FIGS. 18G-H). These neuronal projections from nigra were previously established by the retrograde tracer studies of Bunney and Aghajanian (Brain Res 117 234-435). In addition, on the contralateral side, transduction was observed of several (uninjected) commissural nuclei and their projections (FIG. 12A and 181) providing further evidence of retrograde transport operating with this vector.

### Example 3

### Isolation of Novel Trophic Factors

A VSV-G pseudotyped lentiviral vector system is constructed as described in Example 1, and used to express a cDNA library. A retroviral stock supernatant is produced by a transient method (as described above) and used to transduce primary rat ventral mesencephalic cultures established under low MOI as described in example 1. The expression of a secretable factor that acts as a trophic factor for dopaminergic neurons is determined in these cultures by measuring THE neurons per cm² on grids after 12 or 21 days culture in minimal media (the trophic factor prevents naturally occuring apoptosis). In addition changes in morphology of TH+ neurons are followed (such as more extensive neurite outgrowth and increased cell body size). Similar effects as observed with GDNF are used as a positive control.

### Example 4

### Isolation of Novel Neuroprotective/Survival Factors

A RbG pseudotyped lentiviral vector system is constructed as described in Example 1, and used to express a cDNA library under the control of a dopaminergic specific promoter. A retroviral stock supernatant is produced by a transient method (as described above) and used to transduce TH positive cells in primary rat ventral mesencephalic cultures established as described in example 1. The expression of a factor that acts as a survival/neuroprotective factor for dopaminergic neurons is determined in these cultures by measuring TH+ neurons per cm² on grids 12 days after exposure to 6-OHDA or MPP+. This identifies factors that act intracellularly and have an antiapoptotic effect. The contents of each of the surviving neurons are subsequently specifically amplified by putchclump PCR to determine the sequence of the introduced cDNA. In addition the RNA from such cells is turned into cDNA and amplified by 17 RNA polymerase and the aRNA hybridised to microarrays containing cDNAs obtained from differential display experiments (ie. mRNAs preferentially expressed in dopaminergic neurons). This can also be applied on SN dopaminergic neurons in tissue sections using the technique of laser capture microdissection (Luo et al 1999, as above).

### Example 5

### Screening for Differentiation Factors for Neural Progenitor Cells

Neural progenitor cells are naturally occuring and are the “new hope” for neural transplantation for brain injury and neurodegenerative disease. Human neural progenitors can be obtained commercially (Clonetics). These are neurospheres of subventricular origin that divide when exposed to EGF (originally identified and still worked upon by Canadian company NeuroSpheres). Rodent progenitor cells can also be isolated.

Several groups have tried to differentiate progenitors to dopaminergic neurons but without great success (not one factor identified to date is capable of triggering the TH phenotype on its own). Recent papers demonstrate an unidentified astrocytic soluble factor involved in inducing dopaminergic TH+ phenotype in neural progenitors (Wagner et al (1999) Nat. Biotechnol. 17:653-659; Kawasaki et al (2000) Neuron 28:31-40). If such factor(s) are identified and can induce near 100% dopaminergic differentiation, they will prove very useful for differentiating grafts of neuroprogenitor cells into dopaminergic neurons after transplantation in the adult nervous system (where such inducible factor might not be expressed or expressed at low levels compared to the embryonic brain).

A RbG pseudotyped lentiviral vector system is constructed as described in Example 1, and used to express a cDNA library from E14 embryo mesencephalon.

Dissection of E14 embryos yields mesencephalic cells. At day 3, when these cultures are stable, they are transduced with the retroviral library. Each 1×10⁵ primary mesencephalic cells are incubated with 0.5 ml of virus stock containing 10 μg/ml polybrene. This viral aliquot contains the equivalent of 200 transducing units (cDNAs). As this necessitates a large number of cultures (5000) the viral stock media needs to be appropriately diluted and frozen and used with sequential culture batches till the screening of the entire library is complete. After 8 hours, 0.5 ml of fresh growth medium is added to the culture and incubated overnight. Next day the cultures are refed and allowed to continue till day 12 when the cells will be stained for TH and counted. Where a significant increase in TH+ cell numbers is observed genomic DNA is isolated and cDNAs are amplified from small amounts (10 ng) of genomic DNAs by PCR using retroviral vector primers and sequenced. Chosen candidates are transfected into cells (293) and conditioned media is then used to reconfirm the result on fresh mesencephalic cultures thus purifying the neurotrophic factor.

In an alternative approach, the library is transduced into HeLa cells, selected for antibiotic-resistance and split into pools of 200 HeLa cells/cDNA clones. (sub-libraries) which are subsequently co-cultured with the neurons where they produce and secrete factors. Where an effect is seen, clones are selected and subjected to limit dilution clones, in order to isolate the cell of interest. The experiment is repeated with conditioned media from the single done to further confirm the effect.

With low mois needed and efficiencies of only 20%, most cells will harbour only a single retrovirus and only less than 10% of the cells might have multiple integrations (Onishi et al 1996).

Once a clone is isolated it can be compared to GDNF (i.e. GDNF expressed from the same vector system) using a survival assay or by measuring the extent to which it blocks the effect (for example, the apoptosis of TH+ neurons) of a neurotoxin (MPTP or 6-OHDA) on these cultures.

### Example 6

### Gene Transfer to Hippocampus Using VSV-G and rabies-G Pseudotyped EIAV Vectors

To test if VSV-G and rabies-G pseudotyped EIAV vectors exhibit similar transduction properties to those observed when injected into the basal ganglia, these vectors are stereotactically injected into the right anteriodorsal hippocampus of rats. In the case of the VSV-G pseudotyped vectors, this leads to strong transduction of neurons in the subiculum and to a lesser extent in the CA1 pyramidal cell layer (FIGS. 19A and B). Cells with neuronal morphology within the stratum oriens are also stained while some glial transduction is observed within the corpus callosum. In addition, anterograde transport of β-gal is observed, resulting in weak staining of axon fibers projecting to stratum moleculare (FIG. 19B) and in few fibers projecting to septum (FIG. 19C).

By contrast, injections of rabies-G pseudotyped EIAV vectors into the hippocampal region leads to strong β-gal staining of CA1 and CA3 pyramidal neurons within the stratum pyrimidale of the rostal hippocampus. This become restricted to the CA1 region in caudal aspects, and some staining is also observed in the CA4 pyramidal cell layer (FIGS. 19D-F). Apical dendrites and axons of CA1 neurons are strongly stained. β-gal staining within the subiculum and corpus callosum is observed (FIG. 19F). Retrograde transport of the viral vector and transduction of distal neurons projecting to the area of viral delivery results in strong staining of the medial forebrain bundle nuclei in the lateral hypothalamus and in the vertical limb of the diagonal band of Broca (with axons projecting to the mediodorsal septal area and to the hippocampus via the fimbia of the fornix) (FIG. 19H), supramammillary hypothalamic nuclei and thalamic nuclei (laterodorsal, anterodorsal and anteroventral nuclei) (FIG. 19G) (Segal (1974) Brain Res 78 1-15). Staining of the contralateral hippocampus is probably due to viral vector leakage during the injection along this folded structure, producing an identical but weaker pattern of staining on that side.

### Example 7

### Gene Transfer to Spinal Cord Using VSV-G or rabies-G Pseudotyped EIAV Vectors

Methods

Intraspinal Injection

For intraspinal injection anesthetized 2 month old rats are placed in a stereotaxic frame and their spinal cords are immobilized using a spinal adaptor (Stoelting Co., IL, USA) and injected into the lumbar spinal cord following laminectomy with 1 μl of pONY8Z vector pseudotyped with rabies-G (n=3) or VSV-G (n=3) (6×10⁸ T.U./ml) at one site. Injections, controlled by an infusion pump (World Precision Instruments Inc., Sarasota, USA), are at 0.1 μl per minute through a 10 μl Hamilton syringe fitted with a 33 gauge needle. Following injection, the needle is left in place for 5 minutes before being retrieved. Two weeks following virus injection, rats receive fluorogold (FG) administration. The sciatic nerve is exposed at mid-thigh level and cut 5 mm proximal to the nerve trifurcation. A small cup containing a 4% w/v fluorogold (FG) solution in saline is placed on the proximal segment of the transected nerve. Five days after application of FG the animals are perfused transcardially with 4% w/v paraformaldehyde. The lumbar spinal cord is dissected out and analysed by immunohistochemistry and X-gal reaction. The number of FG and β-gal double-labelled motoneurons is counted 3 weeks after injection of the viral vector. In addition, brains from these animals are also removed and 50 μm coronal sections are stained in X-gal solution as described above.

Intramuscular Injection

For intramuscular delivery, pONY8Z vectors are injected unilaterally in exposed gastrocnemius muscle with a microsyringe fitted with a 30-gauge needle (Hamilton, Switzerland). Two groups of rats are injected: the first group (n=3) received pONY8Z pseudotyped with rabies-G and the second group of rats (n=3) received pONY8Z pseudotyped with VSV-G (titer of both type of vectors is 3×10 ⁸ T.U./ml). Five sites per animal are injected with 10 μl per site. The solution is infused at speed of approximately 1 μl/min. Two animals from each group are sacrificed 3 weeks post injection. The remaining two rats are anesthetized by an intraperitoneal injection of Hypnorm/Hypnovel solution and FG administration is performed as described previously. Two days after application of FG the animals are sacrificed. All animals are perfused transcardially with 4% w/v paraformaldehyde. Subsequently, the muscles are excised and snap frozen in liquid nitrogen. Spinal cords are excised and cryoprotected in 30% w/v sucrose for 2 days. Transverse and longitudinal sections (25 μm each) of both the muscle and spinal cords are analysed by immunohistochemistry and X-gal reaction. To evaluate the number of transduced neurons, motoneurons, lumbar and thoracic spinal cord are analyzed. The number of β-gal-positive cells double-labelled with NeuN are examined in every third section. The proportion of infected motoneurons is expressed as the percentage of fluorogold back-labeled cells expressing β-gal.

Results

To determine the transduction efficiency of the EIAV vector, intraspinal and intramuscular injections of the P-gal-expressing vectors are performed in the rat. Intraspinal injection of the lentiviral vector is associated only with a mild degree of inflammation, with no significant cell damage (data not shown). All rats tolerated the surgery and lentiviral vector injections without complication. Moreover, coordination and movement of operated animals is unaffected, indicating the absence of functional deterioration following intraspinal injection of the viral vector. Examination of transverse sections of the spinal cord revealed robust reporter gene expression after delivery of both VSV-G and the rabies-G pseudotyped lentiviral vectors (FIG. 20 A, B, H. I). Injection in the lumbar spinal cord leads to β-gal expression in 10,260±513 and in 16,695±835 cells with VSV-G and rabies—pseudotyped vectors, respectively. The rabies-G pseudotyped lentiviral vectors produce a more extensive rostrocaudal spread of expressing cells within the area of viral delivery (lumbar spinal cord) and also in the adjoining thoracic spinal cord.

To identify the phenotype of the cells transduced after intraspinal injections, sections are double-labelled with antibodies to p-gal and either NeuN or GFAP. On average 90% and 80% of the transduced cells are double-labelled with NeuN after VSV-G and rabies-G pseudotyped vector delivery, respectively (FIGS. 20E-G and 20L-N). To assess the percentage of motoneurons expressing the reporter gene, motoneurons are back-labelled with FG (FIGS. 20C-D; 20J-K). The number of FG-positive motoneurons expressing β-gal are evaluated in longitudinal sections of the lumbar spinal cord. Analysis of these sections showed that 52 and 67% of the FG-back labeled motoneurons express β-gal after intraspinal injections of VSV-G and rabies-G pseudotyped EIAV vectors, respectively.

Interestingly, brainstem motoneurons of the tectospinal, vestibulospinal and reticulospinal tracts as well as corticospinal motoneurons located in the layer V of primary motor cortex are retrogradely transduced following intraspinal injection only of the rabies-G lentiviral pseudotyped vector (FIGS. 20O,P). Some spinal commissural interneurons projecting from the contralateral side are also retrogradely transduced (FIG. 20H). Interestingly, retrograde transport of the rabies pseudotyped vector is also found in lumbar spinal motoneurons following injection into the gastrocnemius muscle (FIGS. 20Q-S). Intramuscular injections of rabies-G pseudotyped lentiviral vector led to β-gal expression in 27% of the FG-back labelled motoneurons (approximately 850±90 transduced motoneurons). No muscle transduction is observed with this vector. By contrast, the VSV-G pseudotyped vector transduceds muscle cells surrounding the injection site, at low efficiency, but did not label any cells in the spinal cord (data not shown).

### Example 8

### Minimal Immune Response in CNS After EIAV Vector Injection

Method

Investigation of the Immune Response

Groups of rats received intrastriatal injections of pONY8Z vector pseudotyped either with VSV-G (n=6) or rabies-G (n=6) or an equivalent amount of PBS, using the stereotactic procedure described above. Following euthanasia at 7, 14, and 35 days post injection brains were removed and snap frozen directly in OCT and analysed. Sections (15 μm) were cut onto APES (Sigma) coated slides using a Leica CM3500 cryostat (Milton Keynes, UK). One in every 10 sections were stained with X-gal for 3 hours at 37° C. to identify areas of gene transfer and adjacent sections were selected and stained with monoclonal antibody tissue culture supernatant (TCS) against OX1 (leucocyte common antigen), OX18 (MHC class I), OX42 (complement receptor type 3 on microglia and macrophages) and OX62 (dendritic cells). These antibodies were a kind gift from the MRC Cellular Immunology Unit, Sir William Dunn School of Pathology, Oxford. Sections were incubated overnight in neat TCS and following several washes in PBS, incubated for 1 hour with an HRP conjugated rabbit anti-mouse antibody (Dako, UK). Positive staining was then visualised to a brown color using a diaminobenzidine (DAB) kit (Vector Labs, USA). Sections were counterstained with hematoxylin, dehydrated, cleared and mounted using DePeX (BDH Merck, Poole, UK). X-gal stained sections were counterstained using carminic acid (Sigma, UK) and mounted using Permount (Fisher, USA).

Results

At different time points after gene transfer to the brain (striatum), specific antibody markers are used to detect immune responsive cells at the site of injection, at different time points after vector delivery. In no cases after stereotactic delivery is any adverse brain pathology observed. Control injections with PBS causes negligible immune reaction that consisted of a small infiltration of OX42⁺/ED1⁺ activated macrophages/microglia around the needle tract in the cortex and striatum and also along white matter tracts such as corpus callosum (data not shown). No staining is observed with any of the other markers when PBS is injected. This immunoreactivity declined but is still detectable at 35 days. A similar response with these markers is observed with both viral vector preparations and probably represents the reaction to the injection procedure. In addition, the VSV-G pseudotyped vectors results in an infiltration of OX18+ MHC class I positive cells in the ipsilateral striatum, present at all time points but no leucocytes or dendritic cells are observed at any time point (FIGS. 21A-D). However, the rabies-G vector injection initiated a more acute immune response with infiltrating leucocytes, dendritic cells and MHC class I immunopositive cells into striatum and cortex and also along white matter tracts, meninges and subventicular cell layers (FIGS. 21E-H). Some perivascular cuffing and tightly packed inflammatory cells is observed within the striatum with the OX1 and OX18 markers (FIGS. 21E,F). Reduced levels of response including the absence of dendritic cells are detected at 14 days and decline to background levels by 35 days.

### Example 9

### Gene Transfer into the Sensory Nervous System

a) Injection of the Virus into the Dorsal Horn of the Spinal Cord

The intraspinal injection described in Example 7 is followed except that the site of injection is in the dorsal horn instead of ventral horn. Group of rats are injected with pONY8Z or pONY8.1Z (rabies-G or VSV-G) or equivalent amount of PBS, via a posterior laminectomy within the dorsal horn of the spinal cord. Three injection sites at the lumbar level, separated by 2 mm, are performed. Each rat received 1 μl per site of the viral solution at dorso-ventral coordinate of 0.5 mm. PONY8.1Z (VSV-G) was obtained directly from pONY8.0Z by digestion with Sall and partial digestion with Sapl. Following restriction the overhanging termini of the DNA were made blunt ended by treatment with T4 DNA polymerase. The resulting DNA was then religated. This manipulation results in a deletion of sequence between the LacZ reporter gene and just upstream of the 3′ PPT. The 3′ border of the deletion is nt 7895 with respect to wild type EIAV, Acc. No. U01866. Thus pONY8.1Z does not contain sequences corresponding to the EIAV RREs.

b. Direct Injection of the Virus in the Dorsal Root Ganglia

Dorsal root ganglia (DRG) are surgically exposed by dissecting the musculus multifidus and the musculus longissimus lumborum and by removing the processus accessorius and parts of the processus transversus. EIAV vectors (pONY8 or pONY8.1 version) coding for the reporter gene β-gal are injected directly in the DRG. Subjects receive 0.5 μl of the viral solution per ganglion. All injections are done by using a stereotaxic frame and a Hamilton syringe with 33-gauge needle. The solution is slowly infused at the speed of approximately 0.1 μl/min.

C. Peripheral Administration of the Virus

The procedure of the application of the virus on the skin surface is described in Wilson paper (Wilson et al., 1999). Briefly, the hair is removed from the dorsal of the hindfoot surface. The skin is scarified by using medium-coarse sandpaper. Ten microliters of the viral solution is applied to each foot. The side of pipettor tip is used to spread the virus. The virus is retrogradely transported to the DRG. Subcutaneous injections of the virus in the hindfoot are also perormed. Each rat receives unilateral application or injection of 10 μl viral solution.

d. Direct Injection of the Virus into the Sciatic Nerve

For intranerval injection, the right sciatic nerve of anaesthetized rat is surgically exposed. The nerve is gently placed on to a metal plate and pONY8Z or pONY8.1Z pseudotyped with VSV-G or Rabies-G are injected with a 33-gauge Hamilton syringe over 3 min. The volume injected per rat is 1 μl. The sciatic nerve is anatomically repositioned, and the skin was closed with vicryl 5/0 sutures.

Results

pONY8Z vectors are injected into the dorsal horn in four rats and analysed 5 weeks post-transduction (rabies-G 3.8×10⁸ TU/ml, n=2; VSV-G 1.2×10⁹ TU/ml, n=2). Histological sections from the spinal cord, the dorsal root and DRG are examined at various magnifications. All animals show expression of the marker gene in the immediate neighborhood of the site of injection into the spinal cord. Of 3 rats injected into the spinal cord with pONY8Z rabies-G, 2 show expression of P-gal in Schwann cells. Axonal expression is also seen (FIG. 22AC). The two rats display retrogradely transduced DRG neurons (FIGS. 22D-E). However, in contrast to pONY8Z rabies-G injected rats, no β-gal reactivity is detectable in dorsal root and DRG sections from rats injected with pONY8Z VSV-G.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in biology or related fields are intended to be within the scope of the following claims.

The invention will now be further described by the following numbered paragraphs:

1. The use of a vector system to deliver an EOI to a TH positive neuron, wherein the vector system is or comprises at least a part of a rabies G protein or a mutant, variant, homologue or fragment thereof.

2. The use according to paragraph 1, wherein the vector system is pseudotyped with at least a part of a rabies G protein or a mutant, variant, homologue or fragment thereof.

3. The use according to paragraph 1 or 2, to treat and/or prevent a disease which is associated with the death or impaired function of TH-positive neurons.

4. The use according to paragraph 3, to treat and/or prevent Parkinson's disease.

5. A method for analysing the effect of a POI in a TH positive neuron, comprising the step of using a vector system as defined in paragraph 1 or 2.

6. A method for analysing the function of a gene, or a protein encoded by a gene, in a TH positive neuron, which method comprises the step of inhibiting or blocking the expression of the gene, or causing overexpression of the gene, using a vector system as defined in paragraph 1 or 2.

7. A TH positive neuron transduced with a vector system as defined in paragraph 1 or 2.

8. A genetically manipulated TH positive neuron according to paragraph 7.

9. An immortalised TH positive neuron according to paragraph 8.

10. The use of a genetically manipulated TH positive neuron according to paragraph 8 or

9 in the manufacture of a medicament for use in transplantation.

11. A method for treating and/or preventing a disease in a subject in need of same, said method comprising the step of transplanting a genetically manipulated TH positive neuron according to paragraph 8 or 9 into said subject.

12. The use of a vector system to deliver an EOI to a target site, wherein the vector system travels to the target site by retrograde transport, and wherein the vector system is or comprises at least a part of a rabies G protein or a mutant, variant, homologue or fragment thereof.

13. The use according to paragraph 12, wherein the vector system is pseudotyped with at least a part of a rabies G protein or a mutant, variant, homologue or fragment thereof.

14. The use according to paragraph 12 or 13, which comprises the step of administration of the vector system to an administration site which is distant from the target site, wherein the vector system travels from the administration site to the target site by retrograde transport.

15. The use according to paragraph 14, wherein the administration site is a peripheral site. site, wherein the vector system travels from the administration site to the target site by retrograde transport.

15. The use according to paragraph 14, wherein the administration site is a peripheral site.

16. The use according to any of paragraphs 12 to 15 wherein the vector is administered intramuscularly.

17. The use according to any of paragraphs 12 to 16 to deliver an EOI to a cell in the CNS.

18. The use according to any of paragraphs 12 to 17, to deliver an EOI to a motoneuron.

19. The use according to any of paragraphs 12 to 18, to deliver an EOI to a sensory neuron.

20. The use according to paragraph 19 to treat and/or prevent pain.

21. The use of a vector system as defined in any one of paragraphs 12 to 20, in the manufacture of a pharmaceutical composition to treat and/or prevent a disease in a subject.

22. A method of treating and/or preventing a disease in a subject in need of same, said method comprising the step of using a vector system as defined in any one of paragraphs

12 to 20 to deliver an EOI to a target cell.

23. A method for delivering an EOI to a neuron in the CNS which comprises the following steps:

(i) administration of a vector system to a peripheral site

(ii) retrograde transport of the vector system or part thereof to the neuron wherein the vector system is or comprises at least a part of a rabies G protein or a mutant, variant, homologue or fragment thereof.

We claim:

1. A method of delivering an entity of interest (EOI) to a tyrosine hydroxylase(TH) positive neuron, said method comprising a vector system comprising at least a part of a rabies G protein or a mutant, variant, homologue or fragment thereof, wherein said vector system delivers said EOI to said TH positive neuron.

2. The method according to claim 1, wherein the vector system is pseudotyped with at least a part of a rabies G protein or a mutant, variant, homologue or fragment thereof.

3. The method according to claim 1, further comprising treating and/or preventing a disease associated with death or impaired function of TH positive neurons.

4. The method according to claim 3, wherein said disease is Parkinson's disease.

5. A method for analysing the effect of a protein of interest (POI) in a TH positive neuron, comprising the method of claim 1.

6. A method for analysing a function of a gene, or a protein encoded by a gene, in a TH positive neuron, wherein said method comprises a step of inhibiting or blocking expression of said gene, or causing overexpression of said gene, comprising said method of claim 1.

7. A TH positive neuron transduced with a vector system comprising at least a part of a rabies G protein or a mutant, variant, homologue or fragment thereof.

8. The TH positive neuron of claim 7, wherein the vector system is pseudotyped with at least a part of a rabies G protein or a mutant, variant, homologue or fragment thereof.

9. The TH positive neuron of claim 7, wherein said neuron is immortalised.

10. A pharmaceutical composition comprising said TH positive neuron of claim 7.

11. A method for treating and/or preventing a disease in a subject in need of same, said method comprising transplanting a composition comprising a genetically manipulated TH positive neuron transduced with a vector system comprising at least a part of a rabies G protein or a mutant, variant, homologue or fragment thereof into said subject.

12. A method of delivering an EOI to a target site, said method comprising a vector system comprising at least a part of a rabies G protein or a mutant, variant, homologue or fragment thereof, wherein said vector system travels to said target site by retrograde transport.

13. The method according to claim 12, wherein said vector system is pseudotyped with at least a part of a rabies G protein or a mutant, variant, homologue or fragment thereof.

14. The method according to claim 12, further comprising a step of administrating said vector system to an administration site which is distant from the target site, wherein said vector system travels from said administration site to said target site by retrograde transport.

15. The method according to claim 14, wherein said administration site is a peripheral site.

16. The method according to claim 12, wherein said administering is intramuscular.

17. The method according to claim 12, wherein said target site is a cell in the CNS.

18. The method according to claim 12, wherein said target site is a motoneuron.

19. The method according to claim 12, wherein said target site is a sensory neuron.

20. A method of treating and/or preventing pain comprising the method of claim 19.

21. A pharmaceutical composition in an effective amount for treating and/or preventing a disease in a subject in need of same, said composition comprising a vector system comprising at least a part of a rabies G protein or a mutant, variant, homologue or fragment thereof, wherein said vector system travels to a target site by retrograde transport.

22. A method of treating and/or preventing a disease in a subject in need of same, said method comprising said method of claim 12.

23. A method for delivering an EOI to a neuron in the CNS comprising administrating a vector system to a peripheral site, wherein said vector system comprises at least a part of a rabies G protein or a mutant, variant, homologue or fragment thereof, and retrograde transport of said vector system or part thereof to the neuron.
